(19) United States
(12) Patent Application Publication
(10) Pub. No.: US 2010/0069389 A1
(43) Pub. Date: Mar. 18, 2010

(54) NOVEL FORMS OF REBOXETINE

(75) Inventors: Isabel Kalofonos, Cambridge, MA (US); G. Patrick Stahly, West Lafayette, IN (US); William Martin-Doyle, Newton, MA (US); Dimitris Kalofonos, Cambridge, MA (US); Wei Lai, West Lafayette, IN (US); David T. Jonaitis, Brookston, IN (US); A. Gracia Lityo, San Diego, CA (US)

Correspondence Address: O'BRIEN JONES, PLLC, 8200 Greensboro Drive, Suite 1020A, McLean, VA, 22102, US

(73) Assignee: Bionevia Pharmaceuticals, Inc., Cambridge, MA (US)

(21) Appl. No.: 12/553,564

(22) Filed: Sep. 3, 2009

Related U.S. Application Data
(60) Provisional application No. 61/094,903, filed on Sep. 6, 2008.

(51) Int. Cl. A61K 31/5375; C07D 295/12; A61P 25/24; A61P 25/22

(52) U.S. Cl. 514/239.2; 544/177

(57) ABSTRACT

The invention relates to novel crystalline forms of (2R)-2-[(R)-(2-ethoxyphenoxy)-phenylmethyl]morpholine hydrochloride. In various embodiments, the novel crystalline forms may be substantially enantiopure. The preparation and characterization of the novel crystalline forms of (2R)-2-[(R)-(2-ethoxyphenoxy)-phenylmethyl]morpholine hydrochloride is also described. The invention also relates to pharmaceutical compositions containing novel crystalline forms of (2R)-2-[(R)-(2-ethoxyphenoxy)-phenylmethyl]morpholine hydrochloride, which are useful to treat and/or prevent various conditions such as nervous system and pain disorders.

### CROSS-REFERENCE TO RELATED APPLICATIONS

This application claims priority under 35 U.S.C. §119 to U.S. Provisional Application 61/094,903, filed Sep. 6, 2008, which is incorporated herein by reference.

### TECHNICAL FIELD

The invention relates to novel crystalline forms of (2R)-2-[(R)-(2-ethoxyphenoxy)-phenylmethyl]morpholine hydrochloride, processes for making the novel crystalline forms, pharmaceutical compositions comprising the novel crystalline forms, and methods of treating and/or preventing various conditions by administering the novel crystalline forms.

### BACKGROUND

The compound (2RS)-2-[(RS)-(2-ethoxyphenoxy)-phenylmethyl]morpholine (shown below), referred to herein by its common name “reboxetine,” is a known active pharmaceutical ingredient (API) having beneficial therapeutic activity, for example as an antidepressant:

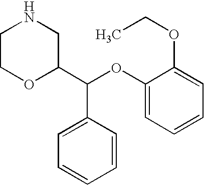

Racemic reboxetine mesylate has a positive indication for the improvement of depression, and is also useful for the treatment of various other nervous system and pain conditions. The preparation of racemic reboxetine mesylate and basic pharmacological activity is described, for example, in Melloni et al., Eur J Med Chem 1984; 19:235; Henegar et al., Org. Process Res. Dev. 2007; 11:346-353; and in U.S. Pat. Nos. 4,229,449; 5,068,433; 5,391,735; and 6,376,711. Therapeutic activity in various conditions has been further described in various references, including, for example, Holm et al., CNS Drugs 1999; 12:65-83; Wong et al., Biological Psychiatry 2000; 47:818-829; Tanum et al., Acta Psychiatr Scand 2000:101(Suppl. 402):37-40; Versiani et al., International Journal of Psychiatry in Clinical Practice 2000; 4:201-208; Versiani et al., Journal of Clinical Psychiatry 2002; 63:31; Hajos et al., CNS Drug Reviews 2004; 10:23-44; Krell et al., Psychosomatics 2005:46:379-384; U.S. Pat. Nos. 6,028,070; 6,046,193; 6,184,222; 6,352,986; 6,391,876; 6,441,038; 6,500,827; 6,586,427; 6,683,114; 6,964,962; 7,241,762; 7,338,953; and U.S. Patent Applications 2004/0048860; 2004/0204411; 2005/0059654; and 2005/0009925.

The separation of racemic reboxetine hydrochloride into its optical isomers (S,S)-reboxetine and (R,R)-reboxetine (also known by the chemical name (2R)-2-[(R)-(2-ethoxyphenoxy)-phenylmethyl]morpholine) is described in, for example Melloni et al., Tetrahedron 1985; 41:1393-1399; GB patent specification 2,167,407; Raggi et al., Electrophoresis 2002; 23:1870-1877; Öhman et al., Journal of Chromatography A 2002; 947; 247-254; and Ficarra et al., Chromatographia 2001; 53:261-265. GB patent specification 2,167,407, Example 12, describes the preparation of a solid from of (R,R)-reboxetine hydrochloride with a melting point of 138-140° C., to be referred to herein as “(R,R)-Reboxetine hydrochloride Form A”.

Therapeutic activity of (S,S)-reboxetine is described in, for example, U.S. Pat. Nos. 6,465,458; 6,610,690; 6,642,235; 6,703,389; 6,987,107; 7,276,503; and 7,317,011; and U.S. Patent Applications 2004/0102440; 2004/0092519; and 2006/0264436.

Although therapeutic efficacy is a primary concern for a therapeutic agent, such as reboxetine, the salt and solid state form (e.g. crystalline or amorphous forms) of a drug candidate can be important to its pharmacological properties and to its development as a viable API. For example, each salt or each crystalline form of a drug candidate can have different solid state (physical and chemical) properties. The differences in physical properties exhibited by a particular solid form of an API, such as a cocrystal, salt, or polymorph of the original compound, can affect pharmaceutical parameters of the API. For example, storage stability, compressibility and dens all of which can be important in formulation and product manufacturing, and solubility and dissolution rates, which may be important factors in determining bioavailability, may be affected. Because these physical properties are often influenced by the solid state form of the API, they can significantly impact a number of factors, including the selection of a compound as an API, the ultimate pharmaceutical dosage form, the optimization of manufacturing processes, and absorption in the body. Moreover, finding the most adequate form for further drug development can reduce the time and the cost of that development.

Obtaining pure crystalline forms, then, is extremely useful in drug development. It may permit better characterization of the drug candidate's chemical and physical properties. For example, crystalline forms often have better chemical and physical properties than amorphous forms. As a further example, a crystalline form may possess more favorable pharmacology than an amorphous form, or may be easier to process. It may also have better storage stability.

One such physical property which can affect processability is the flowability of the solid, before and after milling. Flowability affects the ease with which the material is handled during processing into a pharmaceutical composition. When particles of the powdered compound do not flow past each other easily, a formulation specialist must take that fact into account in developing a tablet or capsule formulation, which may necessitate the use of additional components such as glidants, including colloidal silicon dioxide, talc, starch, or tribasic calcium phosphate.

Another solid state property of a pharmaceutical compound that may be important is its dissolution rate in aqueous fluid. The rate of dissolution of an active ingredient in a patient's stomach fluid may have therapeutic consequences since it can impact the rate at which an orally administered active ingredient may reach the patient's bloodstream.

Another solid state property of a pharmaceutical compound that may be important is its thermal behavior, including its melting point. The melting point of the solid form of a drug is optionally high enough to avoid melting or plastic deformation during standard processing operations, as well as concretion of the drug by plastic deformation on storage (See, e.g. Gould, P. L. Int. J. Pharmaceutics 1986 33 201-217). It may be desirable in some cases for a solid form to melt above about 100° C. For example, inching point categories used by one pharmaceutical company are, in order of preference, +(mp >120° C.), 0 (mp 80-120° C.), and −(mp <80° C.) (Balbach, S.; Korn, C. Int. J. Pharmaceutics 2004 275 1-12).

Active drug molecules may be made into pharmaceutically acceptable salts for therapeutic administration to the patient. Crystalline salts of a drug may offer advantages over the free form of the compound, such as improved solubility, stability, processing improvements, etc. and different crystalline salt forms may offer greater or lesser advantages over one another. However, crystalline salt formation is not predictable, and in fact is not always possible. Moreover, there is no way to predict the properties of a particular crystalline salt of a compound until it is formed. As such, finding the right conditions to obtain a particular crystalline salt form of a compound, with pharmaceutically acceptable properties, can take significant time and effort.

As used herein, the term “polymorph” refers to different crystalline forms of the same compound and other solid state molecular forms, including pseudopolymorphs. The term “pseudopolymorph” as used herein is meant to include hydrates (e.g., water present in the crystalline structure) and solvates (e.g., solvents other than water) of the compound, of both a fixed or stoichiometric and variable nature. Different crystalline polymorphs have different crystal structures due to a different packing of the molecules in the lattice. This results in a different crystal symmetry and/or unit cell parameters which directly influences the physical properties of the form, including X-ray characteristics (both single-crystal and powder diffraction or “XRPD”) of crystals or powders. A different polymorph, for example, will in general diffract at a different set of angles and will give different values for the intensities. Therefore, when available, X-ray techniques can be used to identify different polymorphs, or a solid form that comprises more than one polymorph, in a reproducible and reliable way (S. Byrn et al., Pharmaceutical Solids: A Strategic Approach to Regulatory Considerations, Pharmaceutical research, Vol. 12, No. 7, p, 945-954, 1995; J. K. Haleblian and W. McCrone, Pharmaceutical Applications of Polymorphism, Journal of Pharmaceutical Sciences, Vol. 58, No. 8, p. 911-929, 1969).

Crystalline polymorphic forms are of interest to the pharmaceutical industry, and especially to those involved in the development of suitable dosage forms. If the polymorphic form is not held constant during clinical or stability studies, the exact dosage form used or studied may not be comparable from one lot to another. It is also desirable to have processes for producing, a compound with the selected polymorphic form in high purity when the compound is used in clinical studies or commercial products, since impurities present may produce undesired toxicological effects. Certain polymorphic forms may exhibit enhanced thermodynamic stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies. It is a well-accepted principle that the formation of a new polymorphic form of a compound is totally unpredictable, and until a particular polymorph is prepared, there is no way to know whether it exist, how to prepare it, or what its properties might be.

A crystalline form of a compound, including, for example, a polymorph, a crystalline salt of the compound, or a cocrystal containing the compound or its salt form, generally possesses distinct crystallographic and spectroscopic properties when compared to other crystalline forms having the same chemical composition. Crystallographic and spectroscopic properties of a particular form may be measured by XRPD, single crystal X-ray crystallography, solid state NMR spectroscopy, e.g. ¹³C CP/MAS NMR, or Raman spectroscopy, among other techniques. A particular crystalline form of a compound, of its salt, or of a cocrystal, often also exhibits distinct the al behavior. Thermal behavior can be measured in the laboratory by such techniques as, for example, capillary melting point, TGA, and DSC.

Many organic compounds can exist as optically active forms, i.e., they have the ability to rotate the plane of plane-polarized light. In describing an optically active compound the prefixes R- and S-, and D- and L-, are used to denote the absolute configuration of the molecule about its chiral center(s). The prefixes d- and l-, or (+)- or (−)-, designate the sign of rotation of plane-polarized light by the compound, with l- or (−)-meaning that the compound is levorotary. In contrast, a compound prefixed with d- or (+)-is dextrorotatory. There is no correlation between nomenclature for the absolute stereochemistry and for the rotation of light by an enantiomer. By way of example, d-lactic acid is the same as (−)-lactic acid, and l-lactic acid is the same as (+)-lactic acid. For a given chemical structure, each of a pair of enantiomers is identical except that they are non-superimposable mirror images of one another. In general, enantiomers have identical properties in a symmetrical environment, although their properties may differ in an unsymmetrical environment. A mixture of enantiomers is often called an enantiomeric, or racemic, mixture, or a racemate.

Stereochemical purity may also be important in the pharmaceutical field, where many of the most often prescribed drugs exhibit chirality. For example, the L-enantiomer of the beta-adrenergic blocking agent, propranolol, is known to be 100 times more potent than its D-enantiomer. Additionally, optical purity may be important in the pharmaceutical drug field because certain isomers have been found to impart a deleterious effect, rather than an advantageous or inert effect. For example, it is believed that the D-enantiomer of thalidomide is a safe and effective sedative when prescribed for the control of morning sickness during pregnancy, whereas its corresponding L-enantiomer is believed to be a potent teratogen.

When two chiral centers exist one molecule, there are four possible stereoisomers: (R,R), (S,S), (R,S), and (S,R). Of these, (R,R) and (S,S) are an example of a pair of enantiomers (mirror images of each other), which typically share chemical properties and melting points just like any other enantiomeric pair. The mirror images of (R,R) and (S,S) are not, however, superimposable on (R,S) and (S,R). This relationship is called diastereoisomeric, and the (S,S) molecule is a diastereoisomer of the (R,S) molecule, whereas the (R,R) molecule is a diastereoisomer of the (S,R) molecule.

Currently, reboxetine is available only as a racemic mixture of enantiomers, (S,S) and (R,R) in a 1:1 ratio, and reference herein to the generic name “reboxetine” refers to this enantiomeric, or racemic, mixture of reboxetine mesylate. Racemic reboxetine mesylate is commercially sold under the trade names of EDRONAX™, PROLIFT™, VESTRA™, IRENOR™, and NOREBOX™. As previously noted, reboxetine has been shown to be useful in the treatment of human depressive disorders. Administration of racemic reboxetine, however, can result in certain undesirable side effects such as, for example, dizziness, insomnia, lightheadedness, changes in blood pressure, sweating, gastrointestinal disturbances, sexual dysfunction in males, tachycardia, and urinary retention. Various U.S. patents, including U.S. Pat. Nos. 6,465,458; 6,610,690; 6,642,235; 6,703,389; 6,987,107; 7,276,503; and 7,317,011, state that such side effects occur, in part, because racemic reboxetine lacks a sufficiently high selectivity for inhibiting norepinephrine reuptake (mediated by binding to the norepinephrine transporter, or NET). The above-mentioned patents describe the relative pharmacological selectivity of racemic reboxetine, (S,S)-reboxetine, and (R,R)-reboxetine, for the serotonin and norepinephrine reuptake transporters, which are described with the term Kᵢ; a lower value of Kᵢ for the norepinephrine transporter indicates greater binding affinity to the norepinephrine transporter, while a lower value of Kᵢ for the serotonin transporter indicates greater binding affinity to the serotonin transporter (or 5-HTT), Racemic reboxetine exhibits a pharmacological selectivity of serotonin (Kᵢ)/norepinephrine (Kᵢ) of about 81, indicating that racemic reboxetine is 81 times more selective for the norepinephrine transporter than for the serotonin transporter. Similarly, it has been found that (R,R)-reboxetine is about 15 times more selective for the norepinephrine transporter than for the serotonin transporter, while (S,S)-reboxetine is about 12,770 times more selective for the norepinephrine transporter than for the serotonin transporter. The relative affinities of the three compounds are detailed in Table 1, below:

TABLE 1

| | Norepinephrine | Serotonin | |
|---|---|---|---|
| | Transporter | Transporter | Selectivity Ratio |
| | Affinity | Affinity | (Kᵢ Serotonin/Kᵢ |
| Compound | (Kᵢ, NET) | (Kᵢ, 5-HTT) | Norepinephrine) |
| (S,S)-reboxetine | 0.23 ± 0.06 | 2937 ± 246 | 12,770 |
| (R,R)-reboxetine | 7.0 ± 1.7 | 104 ± 43 | 15 |
| Racemic reboxetine | 1.6 ± 0.6 | 129 ± 13 | 81 |

The above-listed patents state that (S,S)-reboxetine's pharmacological selectivity for inhibition of norepinephrine reuptake provides an improved side effect profile in comparison to racemic reboxetine or to (R,R)-reboxetine. However, no supporting data is provided, and it is commonly known in the art that many observed side effects of racemic reboxetine are consistent with being caused by elevated norepinephrine levels, including such symptoms as dry mouth, elevated blood pressure, sweating, tachycardia, and insomnia. Counter to the argument that the more purely noradrenergic (S,S)-enantiomer of reboxetine is superior to the (R,R)-enantiomer from a safety and tolerability standpoint, it is thou t that the severity of the racemic mixture's side effects may be reduced or avoided by the administration of the enantiopure (R,R)-enantiomer of the compound, which has a relatively lower selectivity for the norepinephrine transporter than does the (S,S)-enantiomer. For example, Denolle et al. (Clin Pharmacol Ther. 1999; 66:282-7) report that administration of racemic reboxetine results in greater blood pressure increases and heart rate increases in humans than does administration of (R,R)-reboxetine, suggesting that the cardiovascular side effects of racemic reboxetine mesylate are primarily caused by (S,S)-reboxetine, presumably by that enantiomer's greater noradrenergic selectivity.

In addition, it is also known in the art that reuptake inhibitor agents with more balanced effects on both norepinephrine and serotonin reuptake (i.e., less purely noradrenergic or serotonergic) have more evidence in support of efficacy in various pain conditions than more selective reuptake inhibitors (see, e.g., lyengar et al., J Pharmacol Exp Ther 2004; 311:576-584: Bomholt et al., Neuropharmacology 2005; 48:252-263; Canavero et al., Pain 2004; 107:279; and Jasmin et al. Pain 2003; 106:3-8); it is also widely known that mixed reuptake inhibitors with a balanced profile of norepinephrine and serotonin reuptake inhibition, such as venlafaxine, duloxetine, desvenlafaxine, and milnacipran, have demonstrated strong efficacy in depression and a wide variety of additional nervous system and pain conditions. Accordingly, it is thought that (R,R)-reboxetine may demonstrate improved efficacy in various nervous system and pain conditions in comparison to (S,S)-reboxetine, given (R,R)-reboxetine's more balanced pharmacological profile.

As used herein, the statement “(R,R)-enantiomer substantially free of the (S,S)-enantiomer” is meant to describe, for example, a compound that comprises about 80% or more by weight of the (R,R)-enantiomer and contains about 20% or less by weight of the (S,S)-enantiomer, such as greater than about 90% by wet greater than about 95% by weight, and greater than about 99% by weight, based on the total weight of the active ingedient. The term “enantiopure” or “substantially enantiopure” as used herein is meant to include, for example, compounds that comprise about 80% or more, such as about 90% or more, about 95% or more, or about 99% or more, of one enantiomer of the referenced compound.

Although there are ma y procedures known in the art for separating enantiomers or synthesizing enantiopure compounds, not all procedures work for all compounds. Thus, much time and effort is often spent devising a procedure that is effective for separating or synthesizing the desired enantiomer of many compounds.

In the following description, various aspects and embodiments of the invention will become evident. In its broadest sense, the invention could be practiced without having one or more features of these aspects and embodiments. Further, these aspects and embodiments are exemplary. Additional objects and advantages of the invention will be set forth in part in the description which follows, and in part will be obvious from the description, or may be learned by practicing of the invention. The objects and advantages of the invention will be realized and attained by means of the elements and combinations particularly pointed out in the appended claims.

### SUMMARY

In accordance with various embodiments of the invention and after extensive experimentation, the inventors have discovered novel polymorphic forms of crystalline (R,R)-reboxetine hydrochloride, which may be substantially enantiopure.

The invention in various embodiments also relates to processes of preparing those novel polymorphic forms, pharmaceutical compositions containing them, and their use in the treatment and/or prevention of various conditions including, for example, various nervous system and pain disorders.

As used herein, the term “XRPD” refers to x-ray powder diffraction. The XRPD data disclosed herein were obtained using an Inel XRG-3000 diffractometer equipped with a CPS (Curved Position Sensitive) detector with a 2θ range of 120°. Real time data were collected using Cu—Kα radiation at a resolution of 0.03° 2θ. The tube voltage and amperage were set to 40 kV and 30 mA, respectively. The monochromator slit was set at 1-5 mm by 160 μm. Samples were prepared for analysis by packing them into thin-walled glass capillaries. Each capillary was mounted onto a goniometer head that is motorized to pc spinning of the capillary during data acquisition. Instrument calibration was performed using a silicon reference standard.

As used herein, the term “DSC” refers to differential scanning calorimetry. DSC data disclosed herein were obtained using a TA Instruments differential scanning calorimeter Q2000. The sample was placed into an aluminum DSC pan, and the weight accurately recorded. The pan was non-crimped and the contents heated under nitrogen under the conditions given in the figures. Indium metal was used as the calibration standard.

As used herein, the term “¹H-NMR” refers to proton nuclear magnetic resonance spectroscopy. Solution ¹H NMR data disclosed herein were acquired on a Varian ^{UNITY}INOVA-400 spectrometer (¹H Larmor Frequency=399.8 MHz). The specific parameters of each spectrum are provided on the attached figures.

As used herein, the term “TGA” refers to thermogravimetric analysis. TGA data disclosed herein were obtained using a TA Instruments 2950 thermogravimetric analyzer. Each sample was placed in an aluminum sample pan and inserted into the TG furnace. The heating conditions are shown in the figures. Nickel and Alumel™ were used as the calibration standards.

As used herein, “Raman” refers to Raman spectroscopy. FT-Raman spectra were acquired on an FT-Raman 960 spectrometer (Thermo Nicolet). This spectrometer uses an excitation wavelength of 1064 nm. The samples were prepared for analysis by placing the material in a glass capillary and positioning it in a gold-coated capillary holder. Wavelength calibration was performed using sulfur and cyclohexane.

As used herein, for example with respect to the various analytical techniques described herein and data generated therefrom, the term “substantially” the same as or similar to is meant to convey that a particular set of analytical data is, within acceptable scientific limits, sufficiently similar to that disclosed herein such that one of skill in the art would appreciate that the crystal salt form of the compound is the same as that of the present invention. One of skill in the art would appreciate that certain analytical techniques, such as, for example, XRPD, ¹H-NMR, DSC, TGA, and Raman, will not produce exactly the same results every time due to, for example, instrumental variation, sample preparation, scientific error, etc. By way of example only, XRPD results (i.e. peak locations, intensities, and/or presence) may vary slightly from sample to sample, despite the fact that the samples are, within accepted scientific principles, the same form, and this may be due to, for example, preferred orientation or varying solvent or water content. It is well within the ability of those skilled in the art, looking at the data as a whole, to appreciate whether such differences indicate a different form, and thus determine whether analytical data being compared to those disclosed herein are substantially similar. In this regard, and as is commonly practiced within the scientific community, it is not intended that the exemplary analytical data of the novel polymorphic forms of (R,R)-reboxetine hydrochloride disclosed herein be met literally in order to determine whether comparative data represent the same form as those disclosed and claimed herein, such as, for example, whether each and every peak of an exemplary XRPD pattern of the novel polymorphic forms of (R,R)-reboxetine hydrochloride disclosed herein is present in the comparative data, in the same location, and/or of the same intensity. Rather, as discussed above, it is intended that those of skill in the art, using accepted scientific principles, will make a determination based on the data as a whole regarding whether comparative analytical data represent the same or a different form than the novel polymorphic forms of (R,R)-reboxetine hydrochloride disclosed herein.

It is to be understood that both the foregoing general description and the following detailed description are exemplary and explanatory only and are not restrictive of the invention, as claimed.

### BRIEF DESCRIPTION OF THE FIGURES

FIG. 1 shows an XRPD pattern with two traces of embodiments of Form B (R,R)-reboxetine hydrochloride, taken from the products of Example 1 (top trace) and Example 3 (bottom trace);

FIG. 2 shows an XRPD pattern with two traces of embodiments of Form C (R,R)-reboxetine hydrochloride, taken from the products of Example (bottom trace) and Example 4 (top trace);

FIG. 3 is a DSC thermogram of an embodiment of Form B (R,R)-reboxetine hydrochloride:

FIG. 4 is a DSC thermogram of an embodiment of Form C (R,R)-reboxetine hydrochloride;

FIG. 5 is a TGA profile of an embodiment of Form B (R,R)-reboxetine hydrochloride;

FIG. 6 is a TGA profile of an embodiment of Form C (R,R)-reboxetine hydrochloride;

FIG. 7A is a full ¹H-NMR spectrum of an embodiment of Form B (R,R)-reboxetine hydrochloride;

FIG. 7B is an ¹H-NMR spectrum from 7.65 to 7.25 ppm of an embodiment of Form B (R,R)-reboxetine hydrochloride;

FIG. 7C is an ¹H-NMR spectrum from 7.1 to 6.5 ppm of an embodiment of Form B (R,R)-reboxetine hydrochloride;

FIG. 7D is an ¹H-NMR spectrum from 5.5 to 4.6 ppm of an embodiment of Form B (R,R)-reboxetine hydrochloride;

FIG. 7E is an ¹H-NMR spectrum from 4.4 to 3.56 ppm of an embodiment of Form B (R,R)-reboxetine hydrochloride;

FIG. 7F is an ¹H-NMR spectrum from 3.5 to 2.9 ppm of an embodiment of Form B (R,R)-reboxetine hydrochloride;

FIG. 7G is an ¹H-NMR spectrum from 2.3 to 0.9 ppm of an embodiment of Form B (R,R)-reboxetine hydrochloride;

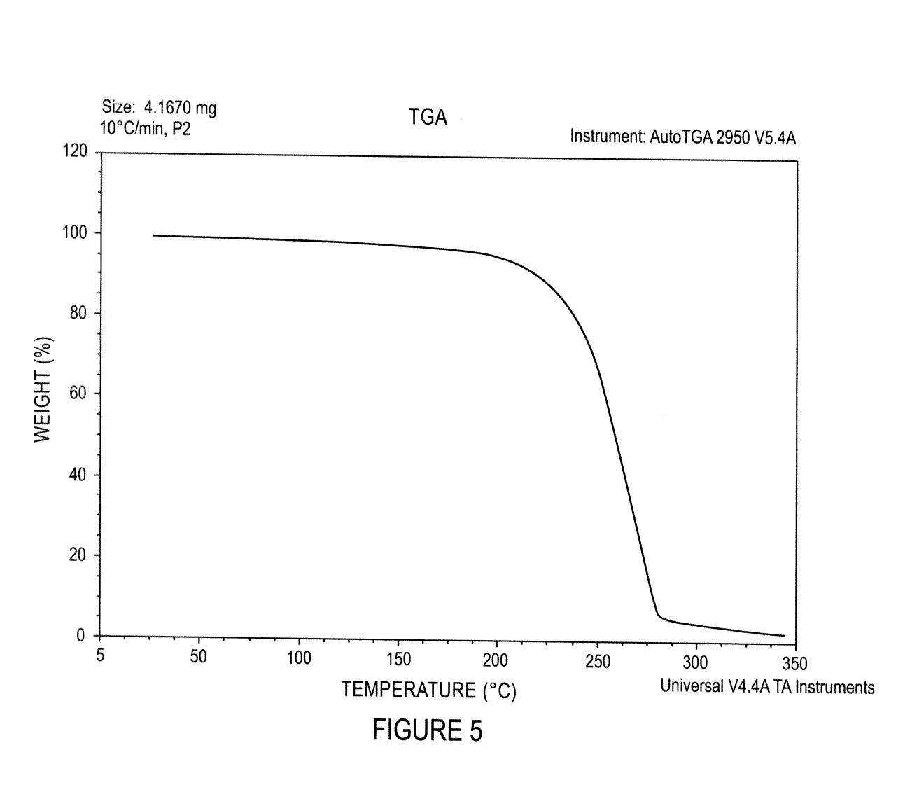

FIG. 5A is a full ¹H-NMR spectrum of an embodiment of Form C (R,R)-reboxetine hydrochloride;

FIG. 5B is an ¹H-NMR spectrum from 7.65 to 7.25 ppm of an embodiment of Form C (R,R)-reboxetine hydrochloride;

FIG. 5c is an ¹H-NMR spectrum from 7.1 to 6.5 ppm of an embodiment of Form C (R,R)-reboxetine hydrochloride;

FIG. 8D is an ¹H-NMR spectrum from 5.60 to 5.15 ppm of an embodiment of Form C (R,R)-reboxetine hydrochloride;

FIG. 8E is an ¹H-NMR spectrum from 4.2 to 3.6 ppm of an embodiment of Form C (R,R)-reboxetine hydrochloride;

FIG. 8F is an ¹H-NMR spectrum from 3.3 to 2.6 ppm of an embodiment of Form C (R,R)-reboxetine hydrochloride;

FIG. 8G is an ¹H-NMR spectrum from 2.2 to 1.0 ppm of an embodiment of Form C (R,R)-reboxetine hydrochloride;

FIG. 9 shows a Raman spectrum with an embodiment of Form B (R,R)-reboxetine hydrochloride (top trace) and an embodiment of Form C (R,R)-reboxetine hydrochloride (bottom trace).

### DESCRIPTION OF EXEMPLARY EMBODIMENTS

The invention relates to novel polymorphic forms of (R,R)-reboxetine hydrochloride. Specifically, two novel polymorphic forms, Form B and Form C, of (R,R)-reboxetine hydrochloride have been discovered. In various exemplary embodiments of the invention, these novel polymorphic forms of (R,R)-reboxetine hydrochloride are in substantially enantiopure form. Exemplary methods of preparation of the novel forms of (R,R)-reboxetine hydrochloride according to various embodiments of the invention are described below in the examples.

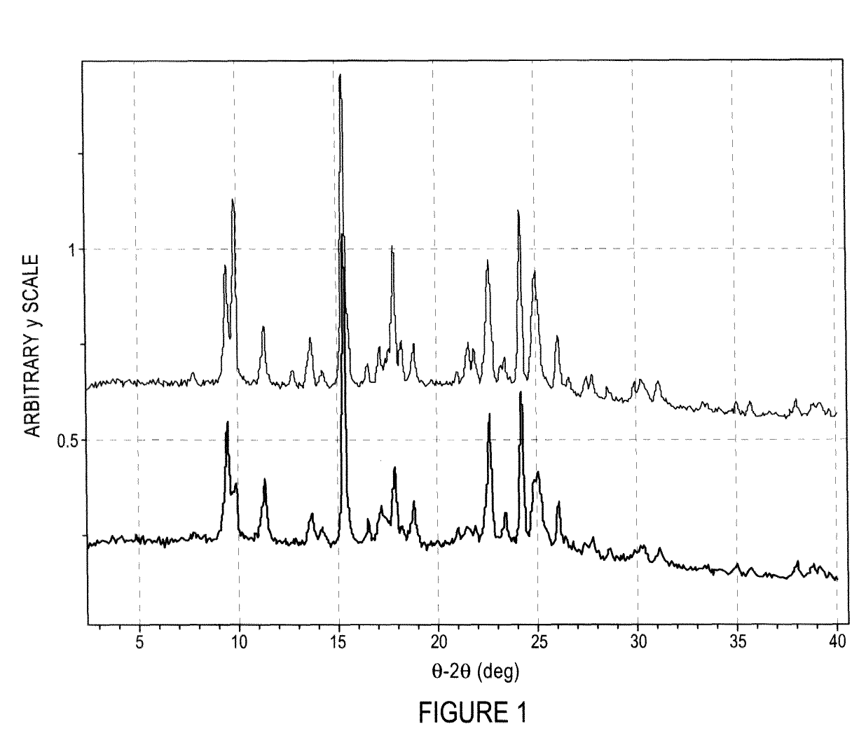

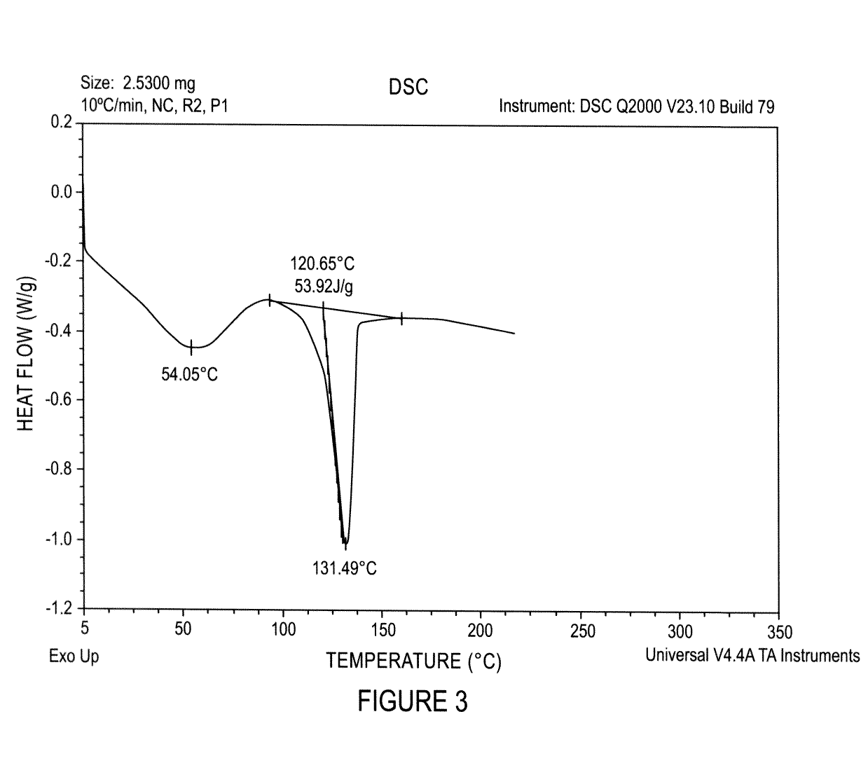

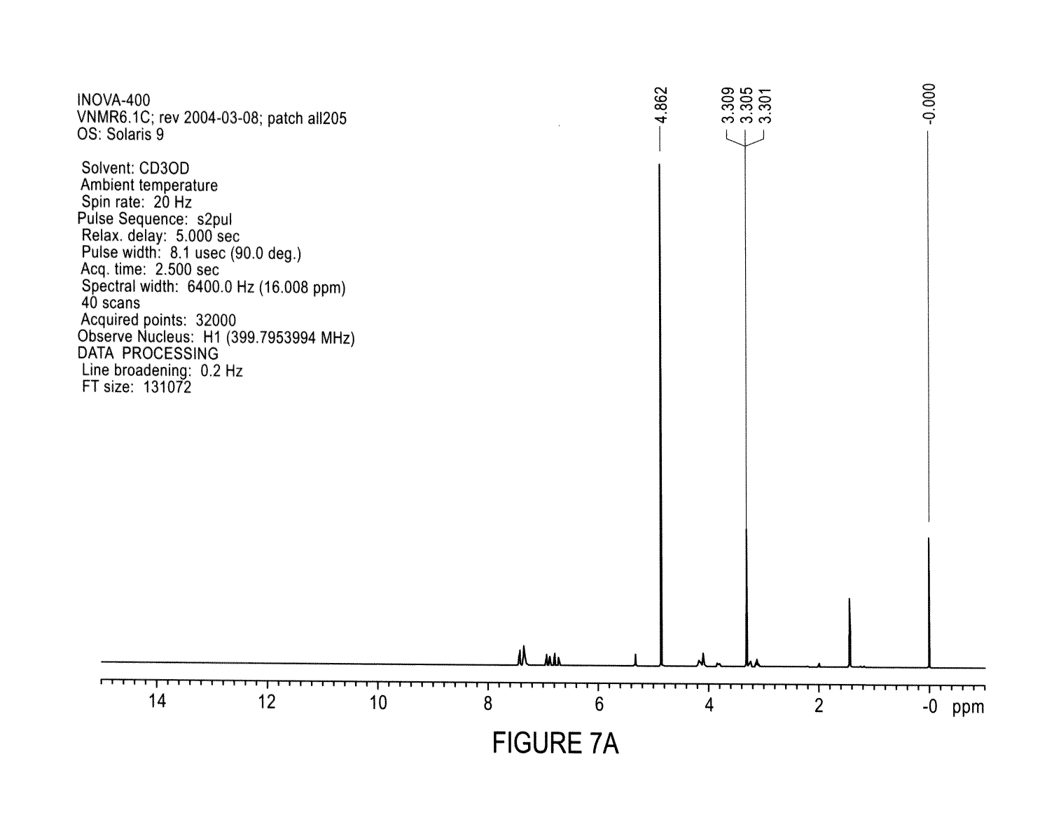

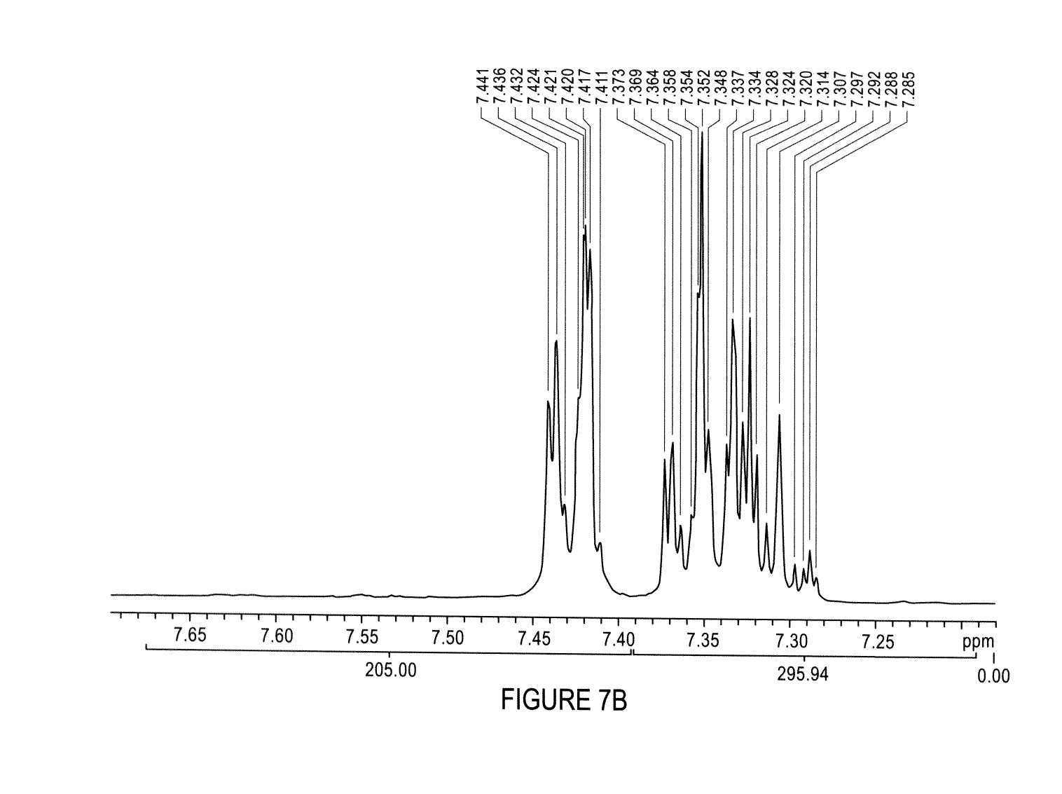

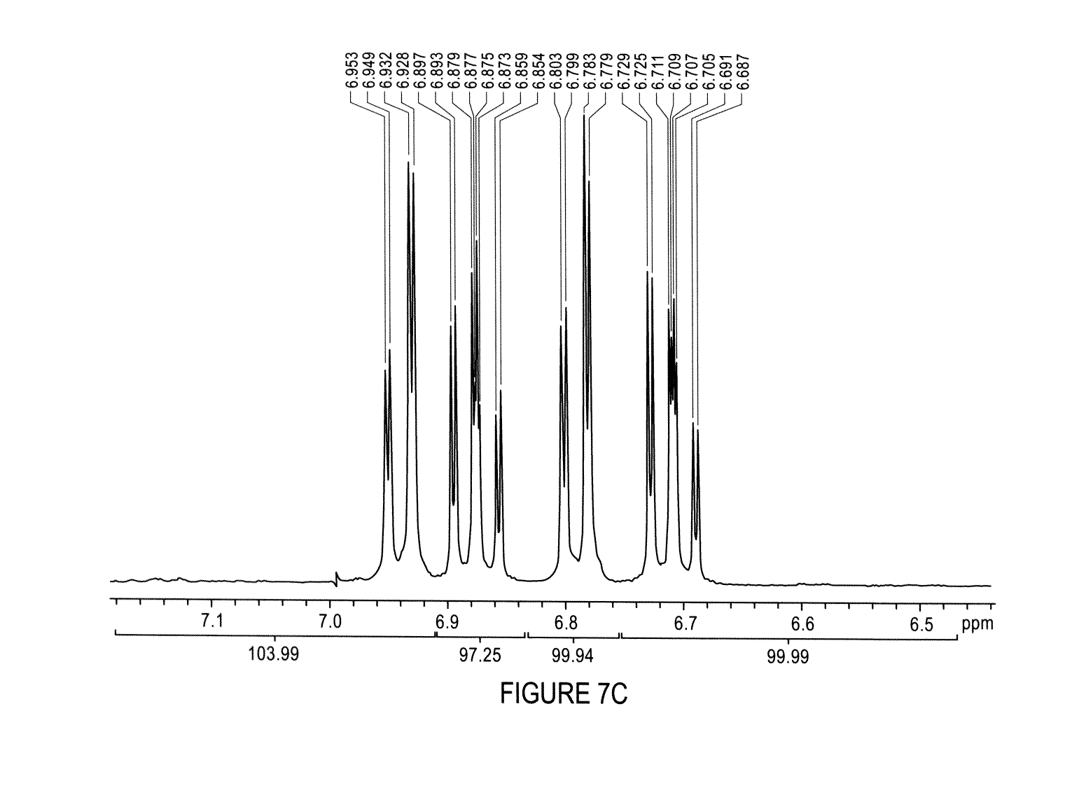

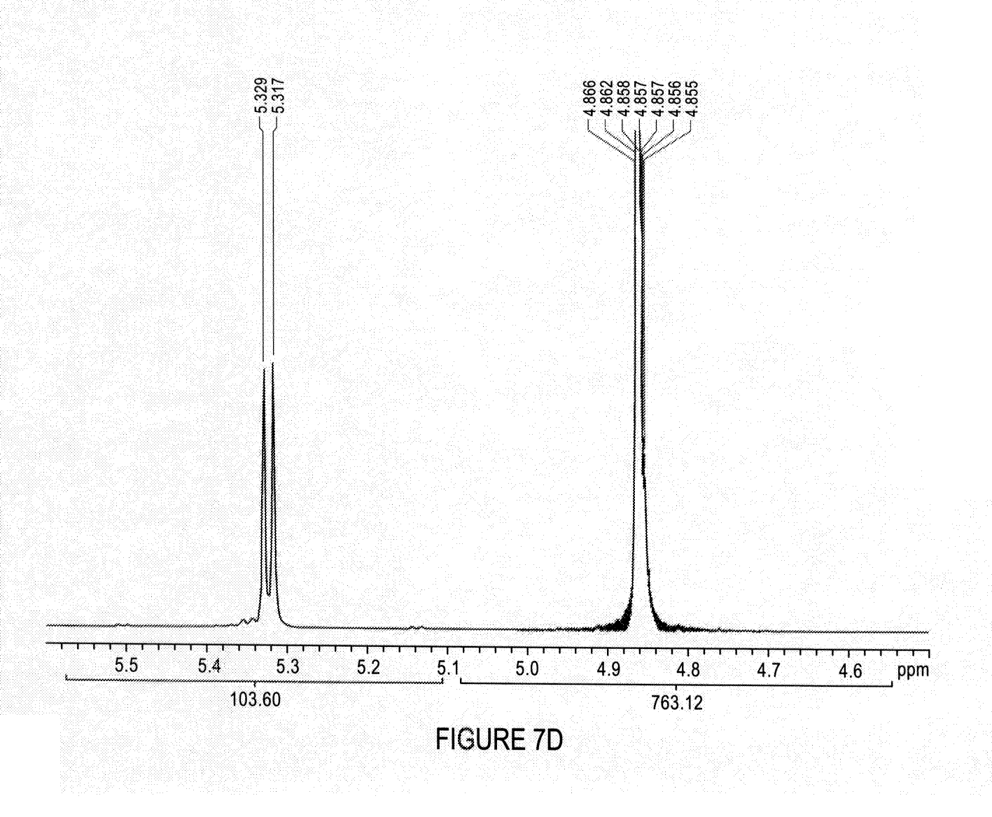

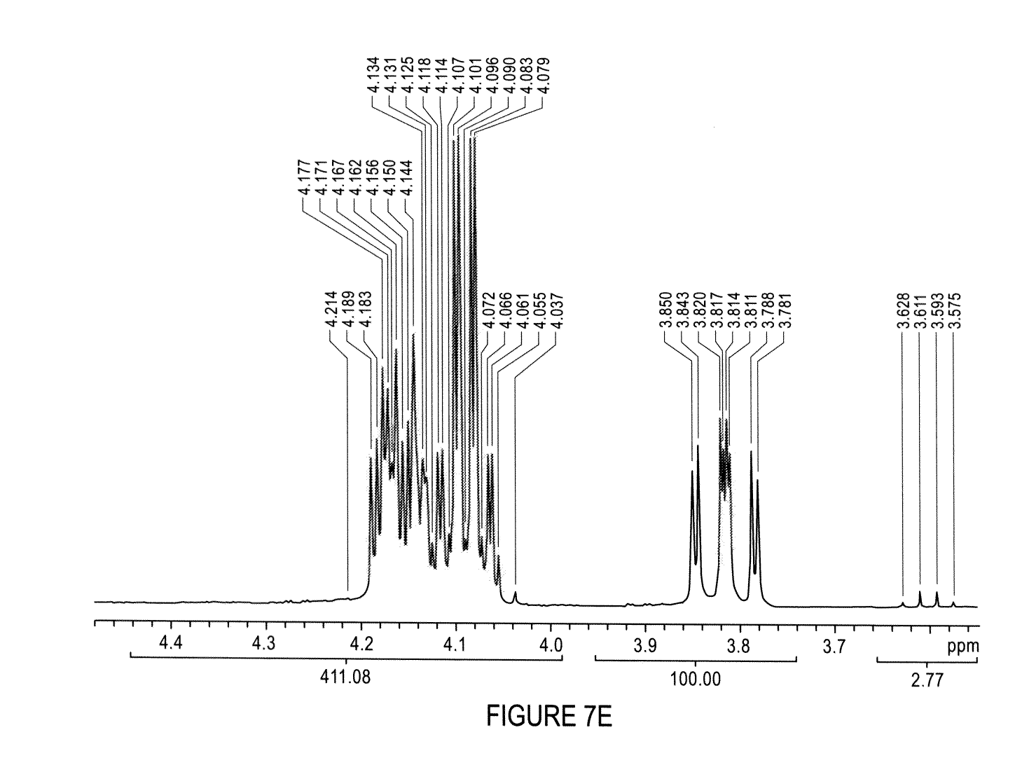

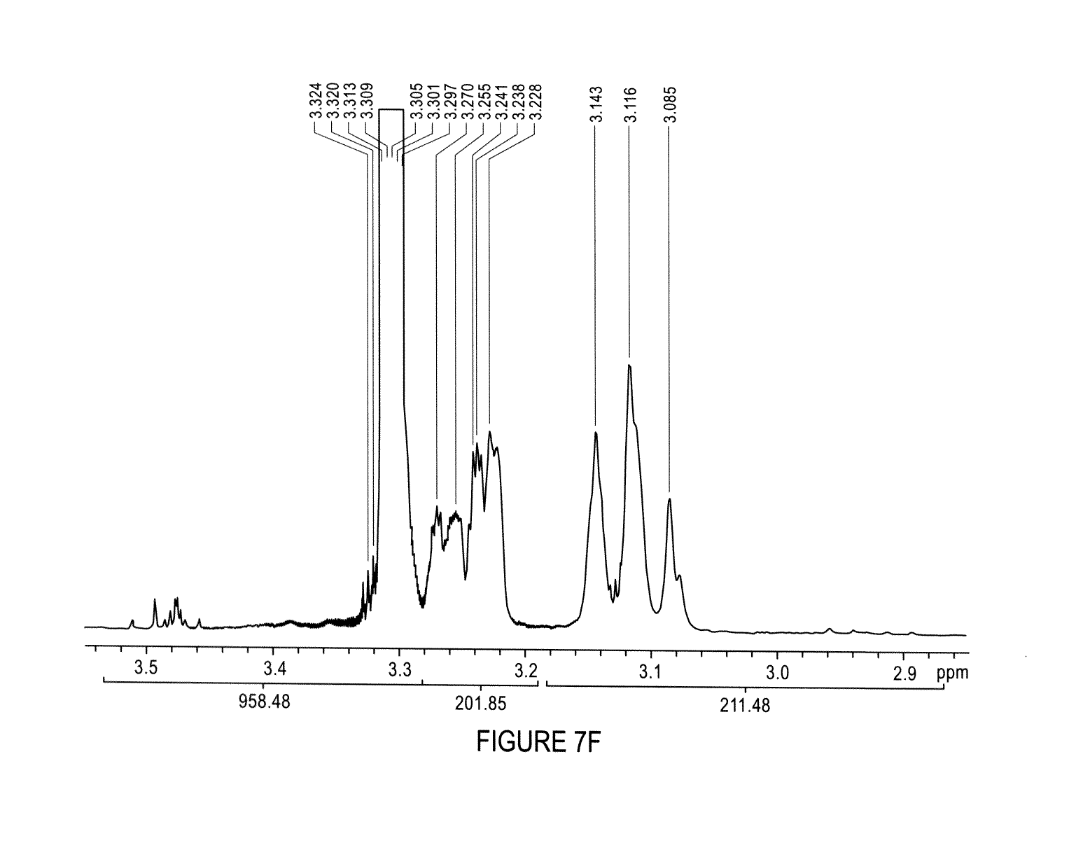

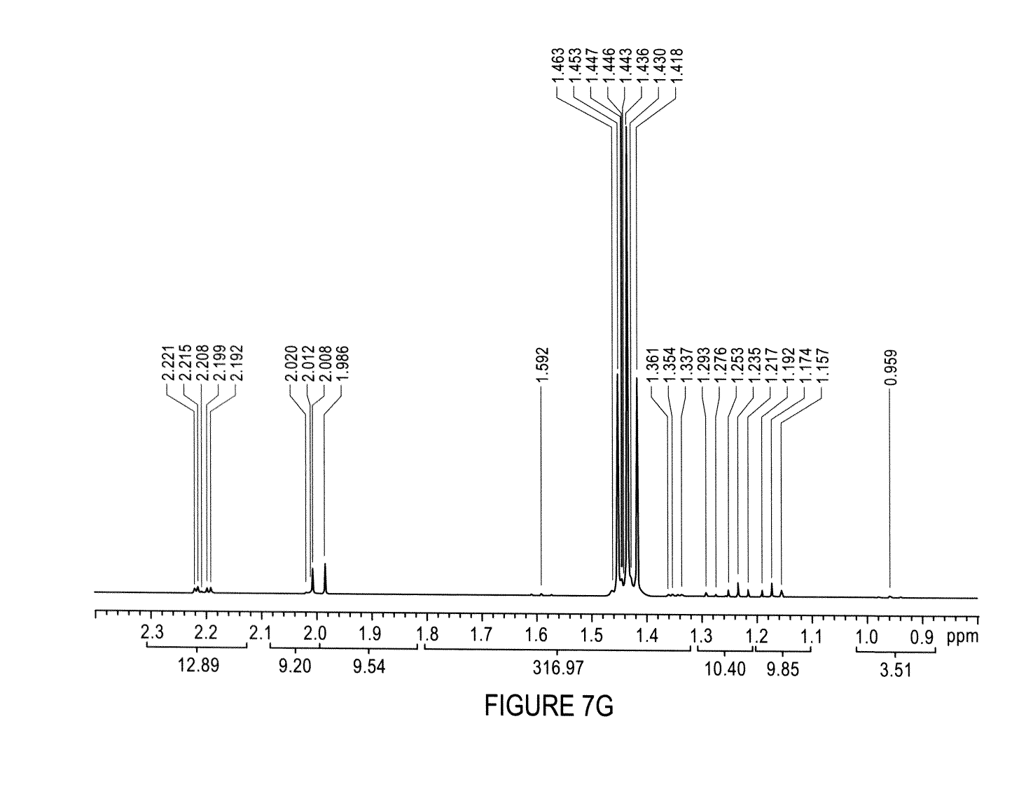

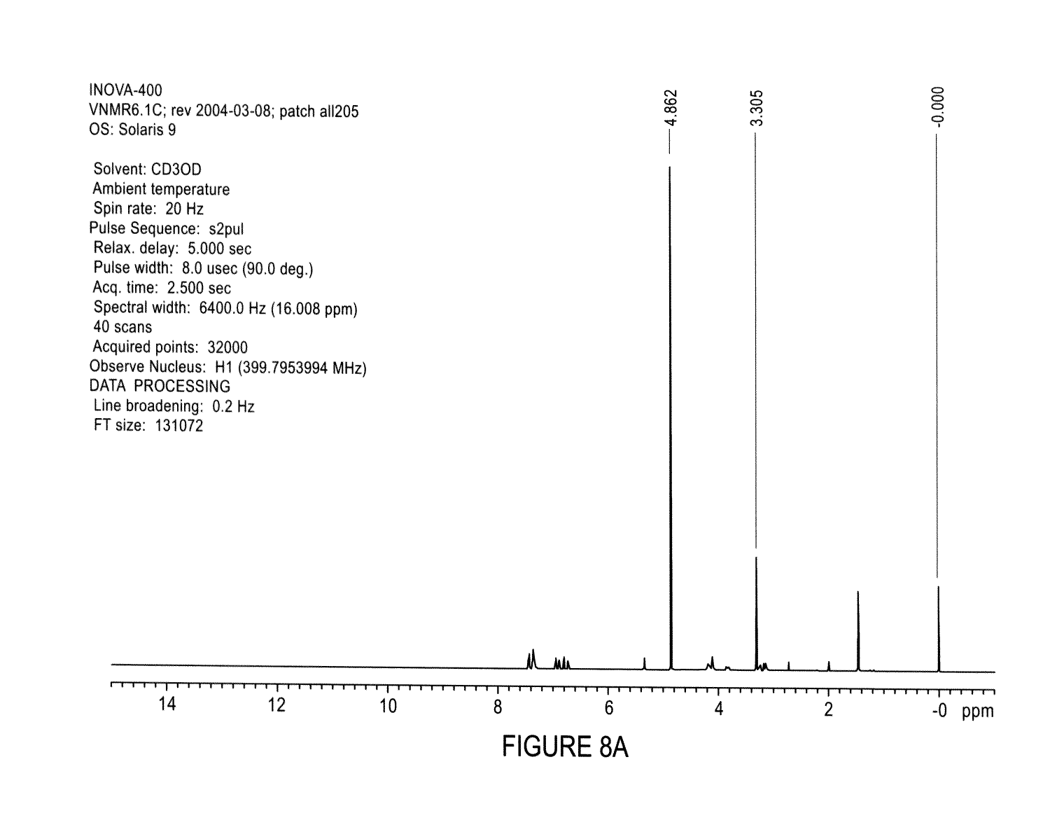

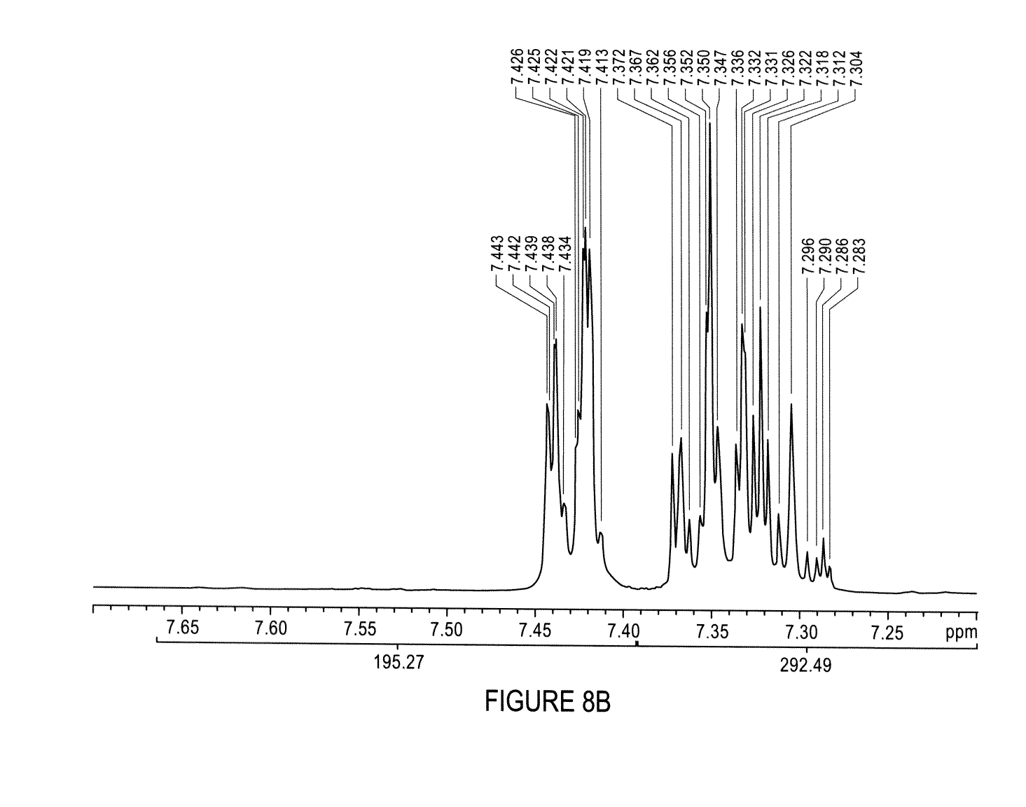

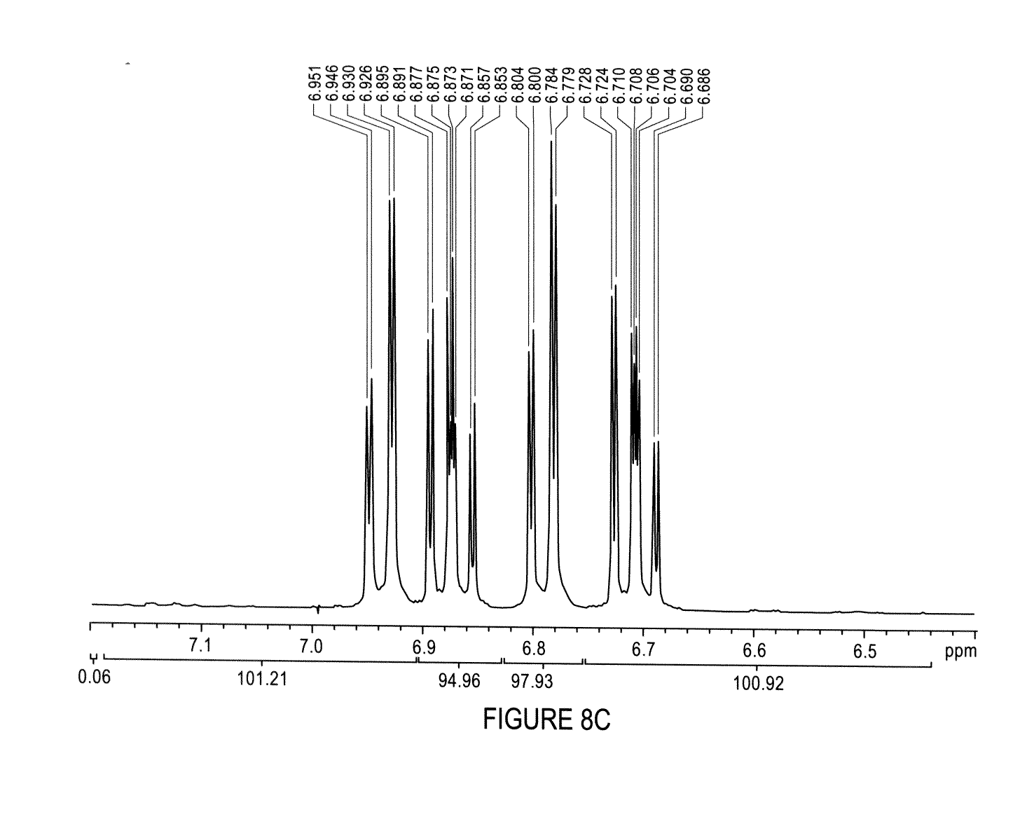

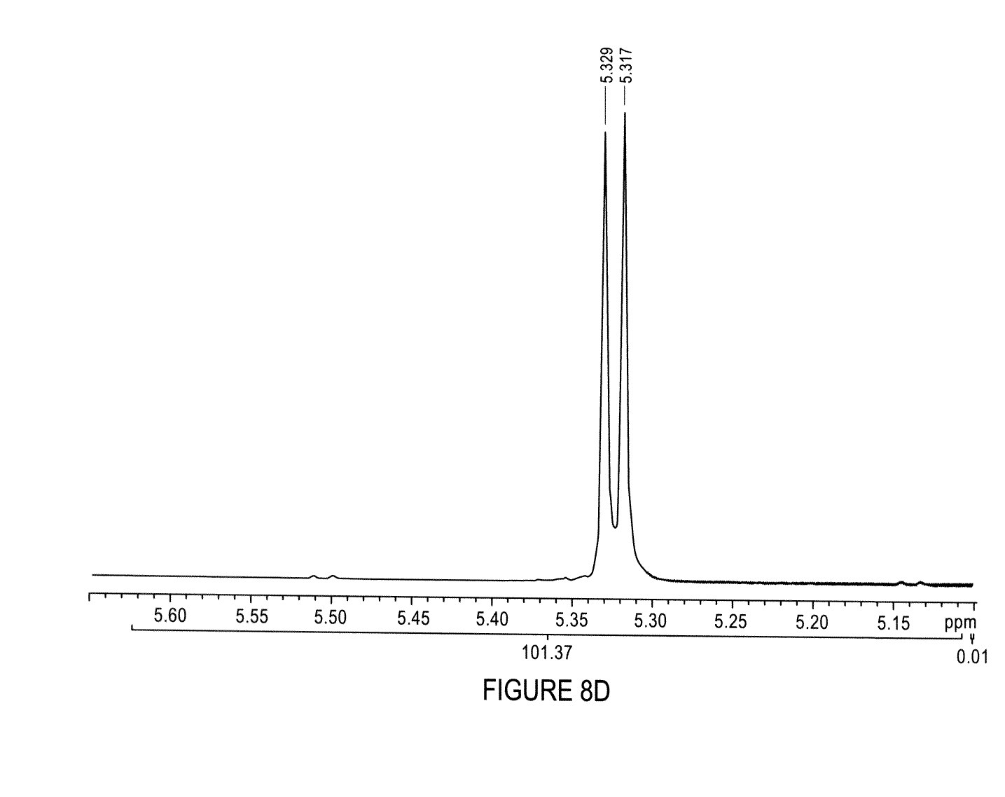

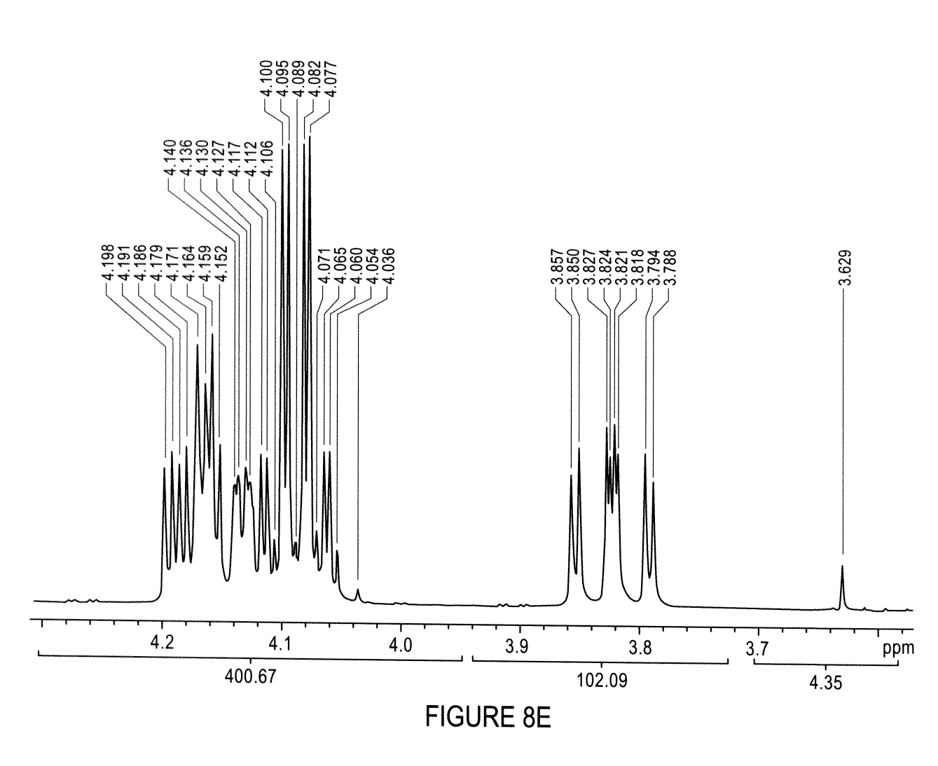

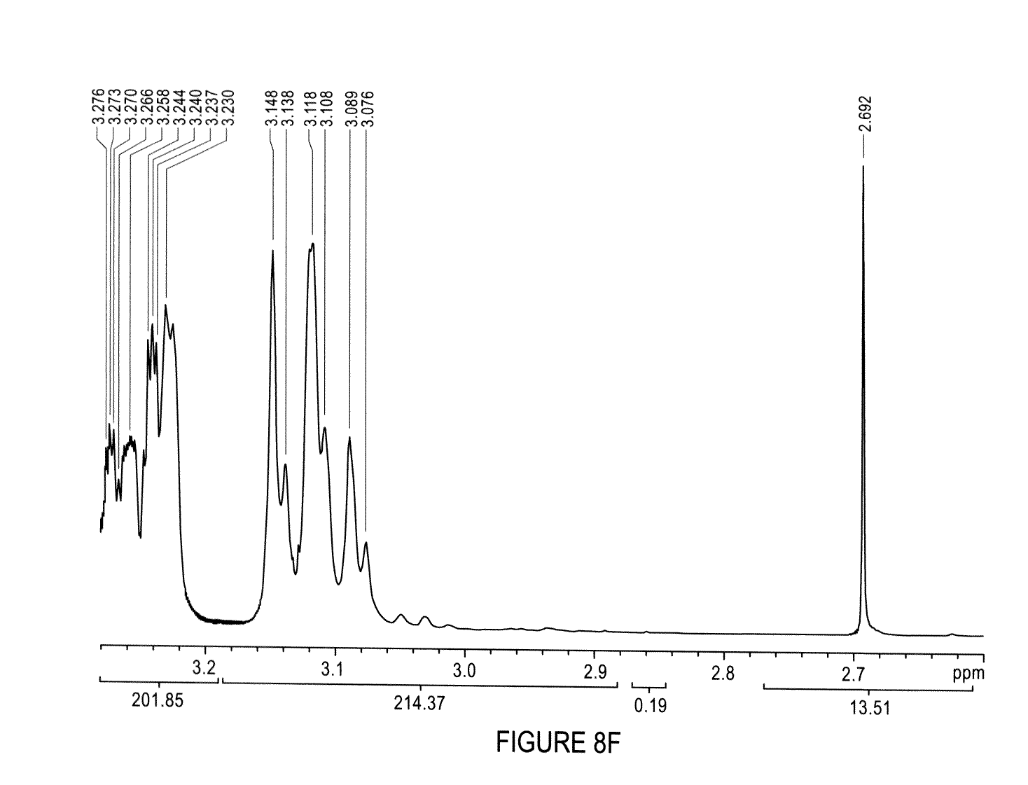

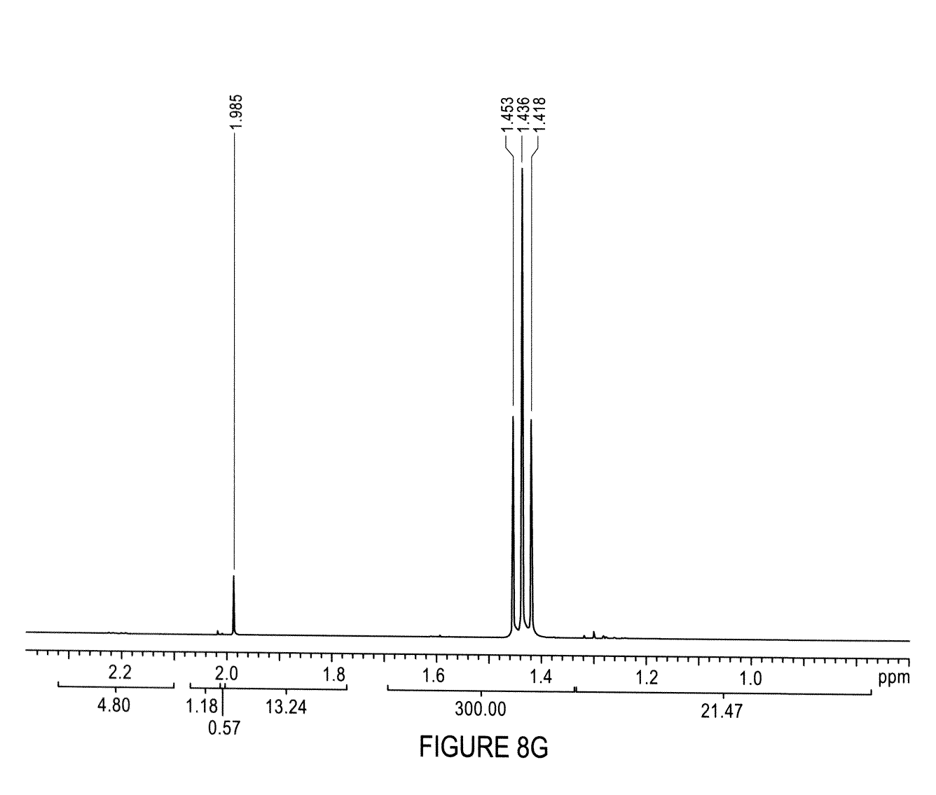

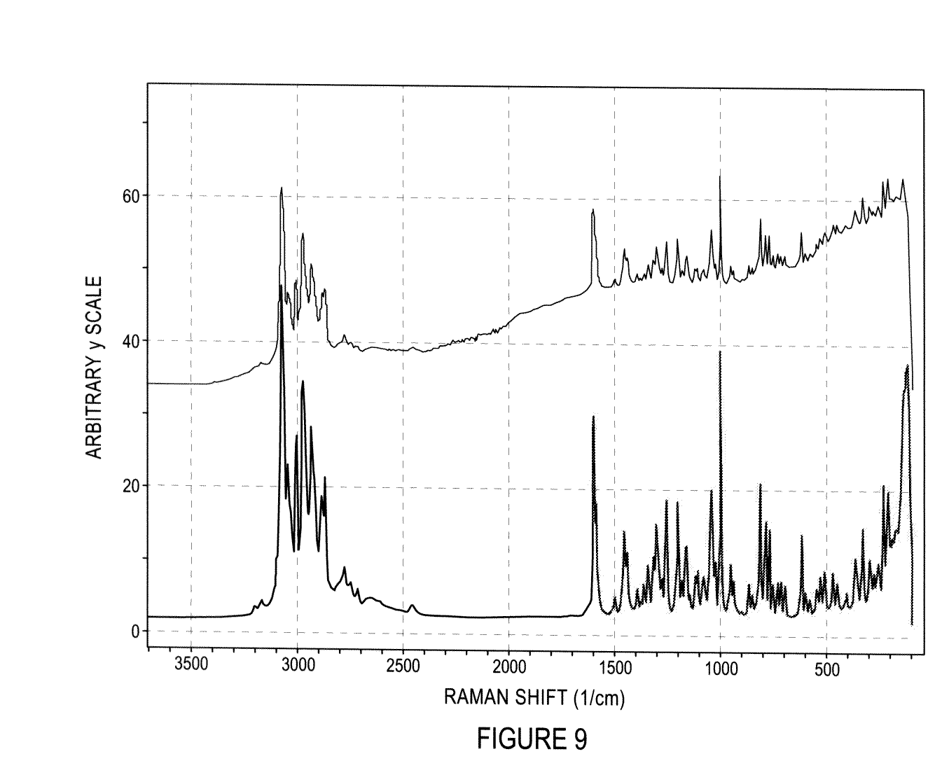

Form B (R,R)-reboxetine hydrochloride is characterized by an XRPD pattern substantially as shown in FIG. 1, a DSC thermogram substantially as shown in FIG. 3, a TGA profile substantially as shown in FIG. 5, and an ¹H-NMR spectrum substantially as shown in FIGS. 7A-7G. An exemplary listing of representative XRPD peaks of an embodiment of Form B (R,R)-reboxetine hydrochloride can be found in Table 2. An exemplary listing of representative NMR data, obtained in CD₃OD, can be found in Table 3.

TABLE 2

| °2θ | d space (Å) | Intensity (%) |
|---|---|---|
| 7.8 ± 0.2 | 11.335 ± 0.29 | 4 |
| 9.5 ± 0.2 | 9.330 ± 0.20 | 39 |
| 9.9 ± 0.2 | 8.935 ± 0.18 | 59 |
| 11.3 ± 0.2 | 7.803 ± 0.14 | 20 |
| 12.8 ± 0.2 | 6.927 ± 0.11 | 5 |
| 13.7 ± 0.2 | 6.473 ± 0.09 | 16 |
| 14.2 ± 0.2 | 6.216 ± 0.09 | 5 |
| 15.3 ± 0.2 | 5.780 ± 0.08 | 100 |
| 16.5 ± 0.2 | 5.373 ± 0.07 | 6 |
| 17.1 ± 0.2 | 5.176 ± 0.06 | 12 |
| 17.9 ± 0.2 | 4.961 ± 0.06 | 45 |
| 18.2 ± 0.2 | 4.872 ± 0.05 | 14 |
| 18.9 ± 0.2 | 4.703 ± 0.05 | 14 |
| 21.0 ± 0.2 | 4.224 ± 0.04 | 4 |
| 21.6 ± 0.2 | 4.120 ± 0.04 | 14 |
| 21.9 ± 0.2 | 4.064 ± 0.04 | 12 |
| 22.6 ± 0.2 | 3.926 ± 0.03 | 41 |
| 23.2 ± 0.2 | 3.831 ± 0.03 | 7 |
| 23.4 ± 0.2 | 3.797 ± 0.03 | 10 |
| 24.2 ± 0.2 | 3.672 ± 0.03 | 55 |
| 25.0 ± 0.2 | 3.563 ± 0.03 | 38 |
| 26.1 ± 0.2 | 3.418 ± 0.03 | 19 |
| 26.6 ± 0.2 | 3.350 ± 0.02 | 5 |
| 27.5 ± 0.2 | 3.242 ± 0.02 | 7 |
| 27.8 ± 0.2 | 3.211 ± 0.02 | 7 |
| 28.6 ± 0.2 | 3.125 ± 0.02 | 4 |
| 29.9 ± 0.2 | 2.987 ± 0.02 | 7 |

TABLE 3

| | | | Coupling | |
|---|---|---|---|---|
| | Peak position | | constant | Number of |
| Protons | (ppm) | Multiplicity | (Hz) | protons |
| impurity | 1.17 | triplet | 7 | low intensity |
| impurity | 1.24 | triplet | 7 | low intensity |
| CH₂CH₃ | 1.44 | triplet | 7 | 3 |
| impurity | 1.99 | singlet | — | low intensity |
| impurity | 2.01 | singlet | — | low intensity |
| impurity | 2.19-2.22 | multiplet | — | low intensity |
| CH₂NCH₂ | 3.09-3.14 | multiplet | — | 2 |
| | 3.23-3.32 | multiplet | — | 2 |
| methanol-d₄ | 3.31 | multiplet | — | — |
| impurity | 3.60 | quartet | 7 | low intensity |
| CH₂OCH | 3.78-3.85 | multiplet | — | 1 |
| CH₂CH₃ | 4.04-4.19 | multiplet | — | 4 |
| methanol-d₄ | 4.86 | singlet | — | — |
| PhCH | 5.32 | doublet | 5 | 1 |
| PhO | 6.69-6.95 | multiplet | — | 4 |
| PhC | 7.29-7.44 | multiplet | — | 5 |

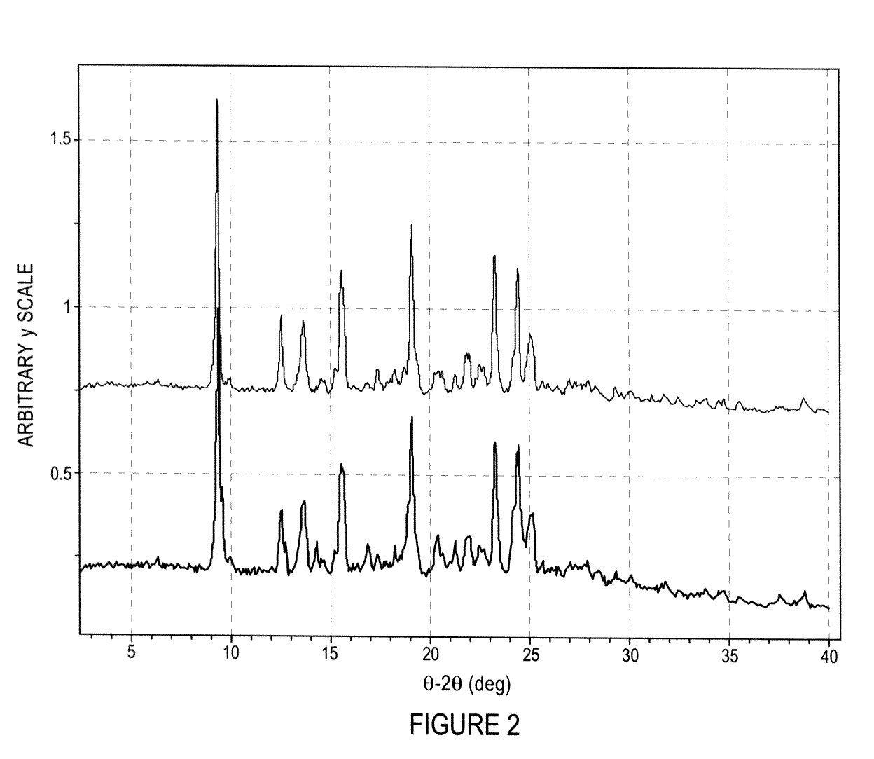

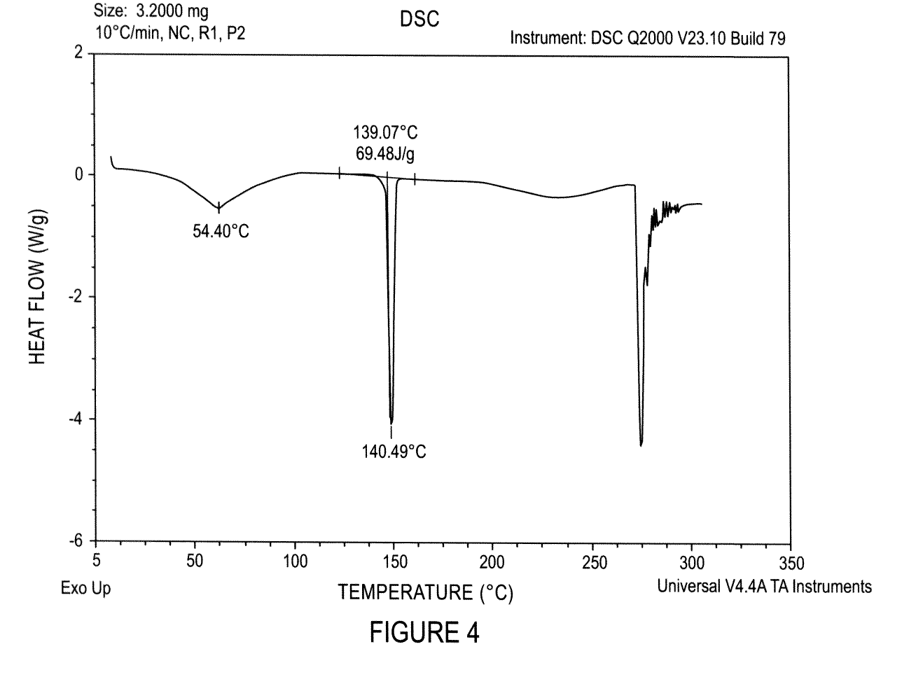

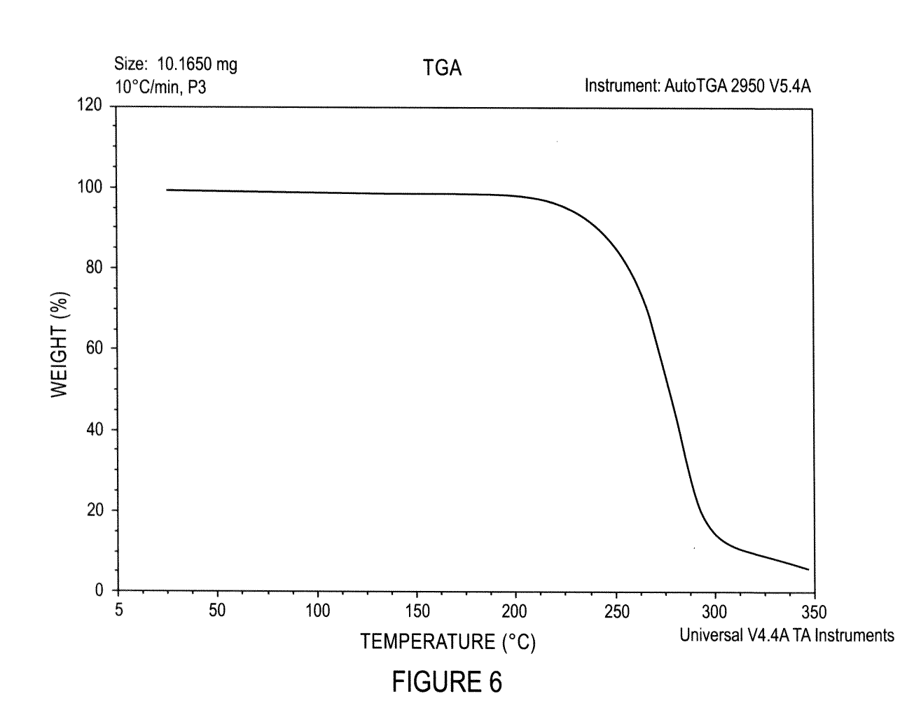

Form C (R,R)-reboxetine hydrochloride is characterized by an XRPD pattern substantially as shown in FIG. 2, a DSC thermogram substantially as shown in FIG. 4, a TGA profile substantially as shown in FIG. 6, and an ¹H-NMR spectrum substantially as shown in FIGS. 8A-8G. An exemplary listing of representative XRPD peaks of an embodiment of Form C (R,R)-reboxetine hydrochloride can be found in Table 4. An exemplary listing of representative NMR data, obtained in CD₃OD, can be found in Table 5.

TABLE 4

| °2θ | d space (Å) | Intensity (%) |
|---|---|---|
| 6.4 ± 0.2 | 13.898 ± 0.44 | 3 |
| 9.4 ± 0.2 | 9.419 ± 0.20 | 100 |
| 10.0 ± 0.2 | 8.881 ± 0.18 | 5 |
| 12.5 ± 0.2 | 7.059 ± 0.11 | 26 |
| 13.6 ± 0.2 | 6.487 ± 0.10 | 25 |
| 14.6 ± 0.2 | 6.088 ± 0.08 | 5 |
| 15.6 ± 0.2 | 5.691 ± 0.07 | 42 |
| 16.1 ± 0.2 | 5.492 ± 0.07 | 3 |
| 16.9 ± 0.2 | 5.259 ± 0.06 | 3 |
| 17.4 ± 0.2 | 5.105 ± 0.06 | 8 |
| 18.2 ± 0.2 | 4.864 ± 0.05 | 8 |
| 19.1 ± 0.2 | 4.644 ± 0.05 | 58 |
| 20.4 ± 0.2 | 4.347 ± 0.04 | 8 |
| 21.3 ± 0.2 | 4.177 ± 0.04 | 6 |
| 21.8 ± 0.2 | 4.070 ± 0.04 | 14 |
| 22.5 ± 0.2 | 3.952 ± 0.03 | 10 |
| 23.2 ± 0.2 | 3.826 ± 0.03 | 48 |
| 24.4 ± 0.2 | 3.645 ± 0.03 | 44 |
| 25.0 ± 0.2 | 3.559 ± 0.03 | 21 |
| 25.6 ± 0.2 | 3.473 ± 0.03 | 5 |
| 26.0 ± 0.2 | 3.434 ± 0.03 | 4 |
| 27.0 ± 0.2 | 3.302 ± 0.02 | 6 |
| 27.9 ± 0.2 | 3.195 ± 0.02 | 6 |
| 28.3 ± 0.2 | 3.151 ± 0.02 | 4 |
| 29.3 ± 0.2 | 3.050 ± 0.02 | 5 |

TABLE 5

| | | | Coupling | |
|---|---|---|---|---|
| | Peak position | | constant | Number of |
| Protons | (ppm) | Multiplicity | (Hz) | protons |
| CH₂CH₃ | 1.44 | triplet | 7 | 3 |
| impurity | 1.99 | singlet | — | low intensity |
| impurity | 2.69 | singlet | — | low intensity |
| CH₂NCH₂ | 3.08-3.15 | multiplet | — | 2 |
| | 3.23-3.28 | multiplet | — | 2 |
| methanol-d₄ | 3.31 | multiplet | — | — |
| Impurity | 3.63 | singlet | — | low intensity |
| CH₂OCH | 3.79-3.86 | multiplet | — | 1 |
| CH₂CH₃ | 4.05-4.20 | multiplet | — | 4 |
| methanol-d₄ | 4.86 | singlet | — | — |
| PhCH | 5.32 | doublet | 5 | 1 |
| PhO | 6.69-6.95 | multiplet | — | 4 |
| PhC | 7.28-7.44 | multiplet | — | 5 |

Pharmaceutical Compositions and Methods of Treatment and/or Prevention

The novel polymorphic forms of (R,R)-reboxetine hydrochloride according to various embodiments of the invention possess substantially the same pharmacological activity as racemic reboxetine mesylate, and are useful for treating and/or preventing various nervous system conditions, including depressive disorders such as major depressive disorder, dysthymic disorder, and depressive disorder not otherwise specified; anxiety disorders (including but not limited to generalized anxiety disorder, panic disorder, obsessive compulsive disorder, social anxiety disorder, social phobia, and post-traumatic stress disorder); minor depressive disorder, recurrent brief depressive disorder; mixed anxiety-depressive disorder; treatment-resistant depression; manic episodes; mixed episodes; hypomanic episodes; bipolar disorders such as bipolar I disorder, bipolar II disorder, cyclothymic disorder, and bipolar disorder not otherwise specified; other mood disorders such as mood disorder due to general medical conditions, substance-induced mood disorder, and mood disorder not otherwise specified; mood disorders with mild, moderate, severe without psychotic features, severe with psychotic features, in partial remission, in full remission, with catatonic features, with melancholic features, with atypical features, with postpartum onset; and depressive episodes associated with bipolar disorders; maintenance treatment of bipolar disorder to prevent recurrence of depressive or manic episodes; psychotic disorders (including schizophrenia, schizoaffective and schizophreniform disorders); seasonal affective disorder; selective serotonin reuptake inhibition (SSRI) “poop out” syndrome (i.e., wherein a patient who fails to maintain a satisfactory response to SSRI therapy after an initial period of satisfactory response); attention-deficit hyperactivity disorder; and attention deficit disorder.

The novel polymorphic forms of (R,R)-reboxetine hydrochloride according to various embodiments of the invention are also useful in treating additional nervous system disorders, including cognitive disorders of various etiologies; age-related cognitive disorder; mild cognitive impairment; postconcussional disorder; mild neurocognitive disorder; dementia (including but not limited to Alzheimer's Disease); dementia with behavioral disturbances; age-associated learning and mental disorders; cognitive disorders due to general medical conditions; personality disorders; borderline personality disorder; behavioral and psychological symptoms of dementia; conduct disorder; autism and autism spectrum disorders; pervasive developmental disorders; sleep disorders (including but not limited to narcolepsy and enuresis); sleep apnea; chronic fatigue syndrome; fatigue associated with various medical and psychiatric conditions; eating disorders (including but not limited to anorexia nervosa and bulimia nervosa): premenstrual dysphoric disorder; sexual dysfunction; overactive bladder; urinary incontinence (including but not limited to stress incontinence, genuine stress incontinence, and mixed incontinence); addictive disorders (including but not limited to those due to alcohol, nicotine, opiates, benzodiazepines, cocaine, sedatives, hypnotics, caffeine, and other psychoactive substances) and withdrawal syndrome; apathy; somatoform disorders (including but not limited to somatization disorder, conversion disorder, hypochondriasis, body dysmorphic disorder, undifferentiated somatoform disorder, and somatoform NOS); inhalation disorders; obesity (i.e. reducing the weight of obese or overweight patients); oppositional defiant disorder; specific developmental disorders; and tic disorders (e.g., Tourette's Disease).

The novel polymorphic forms of (R,R)-reboxetine hydrochloride according to various embodiments of the invention are also useful for treating and/or preventing various pain disorders, including, for example, neuropathic pain and pain associated with nervous system disorders such as, for example, painful diabetic neuropathy, postherpetic neuralgia, trigeminal neuralgia, complex regional pain syndrome I, complex regional pain syndrome II, ischemic neuropathy, phantom limb pain, chemotherapy-induced neuropath HIV-related neuropathy, AIDS-related neuropathy, neuropathic back pain, neuropathic neck pain, carpal tunnel syndrome, other forms of nerve entrapment or nerve compression pain, brachial plexus lesions, other peripheral nerve lesions, neuropathic cancer pain, central neuropathic pain, pain due to multiple sclerosis, post-stroke pain, Parkinson's Disease related central pain, postoperative chronic pain, Guillain-Barre syndrome (GBS), Charcot-Marie-Tooth (CMT) disease, idiopathic peripheral neuropathy, alcoholic neuropathy, other types of neuropathic pain, and other nervous system disorders that have pain as an attendant sip and/or symptom.

The novel polymorphic forms of (R,R)-reboxetine hydrochloride according to various embodiments of the invention are also useful for treating and/or preventing various other types of acute and chronic pain disorders of various etiologies, including, by way of example only, pain resulting from traumatic injury, post-operative pain, post-dental procedure pain, dysmenorrhea, osteoarthritis, rheumatoid arthritis, psoriatic arthritis, fibromyalgia, inflammatory pain, cancer pain, gout, tendonitis pain, bursitis pain, musculoskeletal pain, sports injury-related pain, sprains, strains, pain of osteoporosis, ankylosing spondylitis, headache of various etiologies including but not limited to migraine and tension headache, temporomandibular joint pain, vulvodynia, chronic pelvic pain, interstitial cystitis, myofascial pain syndrome, pain of irritable bowel syndrome, back pain, neck pain, neck-shoulder-arm syndrome, scapulohumeral periarthritis, cervical spondylosis, muscle cramps, idiopathic chronic pain, and visceral pain.

By use of the term “treating” or “alleviating” it is meant decreasing the symptoms, markers, and/or any negative effects of a condition in any appreciable degree in a patient who currently has the condition, and by “preventing” it is meant preventing entirely or preventing to some extent, such as, for example, by delaying the onset or lessening the degree to which a patient develops the condition.

As discussed, additional embodiments of the invention relate to pharmaceutical compositions comprising any amount, such as a therapeutically effective amount, of at least one novel polymorphic form of (R,R)-reboxetine hydrochloride according to various embodiments of the invention, and a pharmaceutically acceptable carrier or excipient. The novel polymorphic forms of (R,R)-reboxetine hydrochloride according to various embodiments of the invention have the same or similar pharmaceutical activity as previously reported for racemic reboxetine mesylate. Pharmaceutical compositions for the treatment and/or prevention of the enumerated conditions or disorders may contain some amount, for example a therapeutically effective amount, of a novel polymorphic form of (R,R)-reboxetine hydrochloride described herein, as appropriate, for treatment of a patient with the particular condition or disorder. As a further example, the amount of a novel polymorphic form of (R,R)-reboxetine hydrochloride in the pharmaceutical compositions may likewise be lower than a therapeutically effective amount, and may, for example, be in the composition in conjunction with another compound or form of reboxetine which, when combined, are present in a therapeutically effective amount. A “therapeutically effective amount” as described herein refers to an amount of a therapeutic agent sufficient to treat, alleviate, and/or prevent a condition treatable and/or preventable by administration of a composition of the invention, in any degree. That amount can be an amount sufficient to exhibit a detectable therapeutic or preventative or ameliorative effect, and can be determined by routine experimentation by those of skill in the art. The effect may include, for example, treatment, alleviation, and/or prevention of the conditions listed herein. The actual amount required, e.g. for treatment of any particular patient, will depend upon a variety of factors including the disorder being treated and/or prevented; its severity; the specific pharmaceutical composition employed; the age, body weight, general health, gender, and diet of the patient; the mode of administration; the time of administration; the route of administration; the rate of excretion of reboxetine; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001.

A pharmaceutical composition according to various embodiments of the invent on may be any pharmaceutical form which contains a novel polymorphic form of (R,R)-reboxetine hydrochloride according, to various embodiments of the invention. Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition according to various embodiments of the invention, that is one having a novel polymorphic form of (R,R)-reboxetine hydrochloride described herein, a carrier may be chosen that maintains the polymorphic, salt, and/or the substantially enantiopure form. In other words, the carrier, in some embodiments, will not substantially alter the polymorphic form, salt form, or the enantiomeric purity of the form of reboxetine described herein. In certain embodiments, the carrier will similarly not be otherwise incompatible with reboxetine itself, crystalline salts of reboxetine, substantially enantiopure crystalline salt forms of reboxetine, or polymorphs of reboxetine hydrochloride according to various embodiments of the invention, such as by producing any undesirable biological effector otherwise interacting in a deleterious manner with any other component(s) of the pharmaceutical composition.

The pharmaceutical compositions according to various embodiments of the invention are optionally formulated in unit dosage form for ease of administration and uniformity of dosage. A “unit dosage form” refers to a physically discrete unit of therapeutic agent appropriate for the patient to be treated. It will be underdressed, however, that the total daily dosage of a novel polymorphic form of (R,R)-reboxetine hydrochloride according to various embodiments of the invention and pharmaceutical compositions thereof will be decided by the attending physician within the scope of sound medical judgment using known methods.

Because the form of reboxetine may be more easily maintained during preparation, solid dosage forms are a preferred form for the pharmaceutical composition of the invention. Solid dosage forms for oral administration may include, for example, capsules, tablets, pills, powders, and granules. In one exemplary embodiment, the solid dosage form is a tablet. The active ingredient may be contained in a solid dosage form formulation that provides quick release, sustained release, or delayed release after administration to the patient. In such solid dosage forms, the active compound may be mixed with at least one inert, pharmaceutically acceptable carrier, such as, for example, sodium citrate or dicalcium phosphate. The solid dosage form may also include one or more of various additional ingredients, including, for example: a) fillers or extenders such as, for example, starches, lactose, sucrose, glucose, mannitol, and silicic acid; b) binders such as, for example, carboxymethylcellulose, alginates, gelatin, polyvinylpyrrolidinone, sucrose, and acacia; c) humectants such as, for example, glycerol; d) disintegrating agents such as, for example, agar, calcium carbonate, potato or tapioca starch, alginic acid, certain silicates, and sodium carbonate; e) dissolution retarding agents such as, for example, paraffin; f) absorption accelerators such as, for example, quaternary ammonium compounds; g) wetting agents such as, for example, cetyl alcohol and glycerol monostearate; h) absorbents such as, for example, kaolin and bentonite clay; and i) lubricants such as, for example, talc, calcium stearate, magnesium stearate, solid polyethylene glycols, and sodium lauryl sulfate. The solid dosage forms may also comprise buffering agents. They may optionally contain opacifying agents and can also be of a composition that they release the active ingredient(s) only, or preferentially, in a certain part of the intestinal tract, optionally, in a delayed manner. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Solid dosage forms of pharmaceutical compositions according to various embodiments of the invention can also be prepared with coatings and shells such as enteric coatings and other coatings well known in the pharmaceutical formulating art.

The novel polymorphic forms of (R,R)-reboxetine hydrochloride according to various embodiments of the invention can be, in one exemplary embodiment, administered in a solid micro-encapsulated form with one or more carriers as discussed above. Microencapsulated forms may also be used in soft and hard-filled gelatin capsules with carriers such as lactose or milk sugar as well as high molecular weight polyethylene glycols and the like.

The novel polymorphic forms of (R,R)-reboxetine hydrochloride according to various embodiments of the invention may also be used in the preparation of non-solid formulations, e.g., injectables and patches, for example of (R,R)-reboxetine. Such non-solid formulations are known in the art. In certain formulations, such as a non-solid formulation, the enantiomeric purity, the salt form, and/or the polymorphic form may, in certain exemplary embodiments, not be maintained. For example, the form may be dissolved in a liquid carrier. In this case, the novel polymorphic forms of (R,R)-reboxetine hydrochloride according to various embodiments of the invention may represent intermediate forms of (R,R)-reboxetine used in the preparation of the non-solid formulation. The novel polymorphic forms of (R,R)-reboxetine hydrochloride according to various embodiments of the invention may provide advantages of handling stability and purity to the process of making such formulations.

In addition, the novel polymorphic forms of (R,R)-reboxetine hydrochloride according to various embodiments of the invention are also useful for administration in combination with other analgesic medication classes, such as strong and weak opioids, NSAIDs, COX-2 inhibitors, acetaminophen, other anti-inflammatories, tricyclic antidepressants, anticonvulsant agents, voltage gated calcium channel blockers, N-type calcium channel blockers, other calcium channel modulators. SNRIs and other monoamine reuptake inhibitors, sodium channel blockers, NK-1 antagonists, NMDA antagonists AMPA antagonists, other glutamate modulators, GABA modulators, CRIMP-2 modulators, TRPV1 agonists, cannabinoids, potassium channel openers, alpha adrenergic agonists, adenosine agonists, nicotinic agonists, p38 MAP kinase inhibitors, corticosteroids, and other analgesic drug classes, and may have a useful dose-sparing effect of lowering the required dosage of the medication used in combination with a novel polymorphic form of (R,R)-reboxetine hydrochloride according to various embodiments of the invention. The novel polymorphic forms of (R,R)-reboxetine hydrochloride according to various embodiments of the invention are therefore also useful for treating or preventing complications or side effects arising from usage of other analgesic medications, including problems with opioids such as dependency, constipation, and respiratory depression. Opioid pain medications can either inhibit or excite the CNS, although it is considered that inhibition is more common. Patients with depressed CNS functions may feel varying levels of drowsiness, lightheadedness, euphoria or dysphoria, or confusion. NSAID pain medications can also induce negative side effects, such as gastrointestinal toxicity or bleeding, renal toxicity, and cardiovascular toxicity. Side effects of other analgesic classes can include sedation, dizziness, anticholinergic effects, dependency, hypotension, and various other adverse effects. These analgesic-induced side effects can manifest themselves when the dosage is increased. Decreasing the dosage of an analgesic or changing medications often helps to decrease the rate or severity of these analgesic-induced side effects. It is thought that a therapeutic amount of a novel polymorphic form of (R,R)-reboxetine hydrochloride according to various embodiments of the invention in combination with a pain agent will reduce the risk of such side effects by reducing the required dosage of the other agent used in combination.

The invention also relates to the treatment and/or prevention of various disorders and/or conditions such as those discussed above, including, for example, various nervous system and pain conditions. The invention provides a method for treating and/or preventing such disorders and/or conditions by administering to mammals, such as a human, a novel polymorphic form of (R,R)-reboxetine hydrochloride as described herein, or a pharmaceutical composition containing the same, in an amount sufficient to treat and/or prevent a condition treatable and/or preventable by administration of a composition of the invention. That amount is the amount sufficient to exhibit any detectable therapeutic and/or preventative or ameliorative effect. The effect may include, for example, treatment and/or prevention of the conditions listed herein. The novel polymorphic forms of (R,R)-reboxetine hydrochloride and pharmaceutical compositions containing them may, according to various embodiments of the invention, be administered using any amount, any form of pharmaceutical composition, and any route of administration effective, e.g. for treatment and/or prevention, all of which are easily determined by those of skill in the art through routine experimentation. After formulation with an appropriate pharmaceutically acceptable carrier in a desired dosage, as known by those of skill in the art, the pharmaceutical compositions can be administered to humans and other mammals by any known method, such as, for example, orally, rectally, or topically (such as by powders or other solid form-based topical formulations). In certain embodiments, the novel polymorphic forms of (R,R)-reboxetine hydrochloride according to various embodiments of the invention may be administered at dosage levels ranging from about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than about 0.001 mg/kg or greater than about 50 mg/kg (for example, ranging from about 50 mg/kg to about 100 g/kg) can also be administered to a subject in certain embodiments of the invention. As discussed above, the amount required for a particular patient will depend upon a variety of factors including the disorder being treated and/or prevented; its severity; the specific pharmaceutical composition employed; the age, body weight, general health, gender, and diet of the patent; the mode of administration; the time of administration; the route of administration; and the rate of excretion of reboxetine; the duration of the treatment; any drugs used in combination or coincidental with the specific compound employed; and other such factors well known in the medical arts. And, as also discussed, the pharmaceutical compositions containing at least one novel polymorphic form of (R,R)-reboxetine hydrochloride as described herein may be administered as a unit dosage form.

Although the present invention herein has been described with reference to various exemplary embodiments, it is to be understood that these embodiments are merely illustrative of the principles and applications of the present invention. Those having skill in the art would recognize that a variety of modifications to the exemplary embodiments may be made, without departing from the scope of the invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from consideration of the specification and practice of the invention disclosed herein. It is intended that the specification and examples be considered as exemplary only, with a scope and spirit being indicated by the claims.

### EXAMPLES

### Example 1

### Preparation of Form B of (R,R)-Reboxetine Hydrochloride

A solution of 138 mg (0.440 mmol) of R,R-reboxetine free base in 2.76 mL of ethyl acetate was treated with 88 μL, of a 1.0 M solution of hydrogen chloride in diethyl ether (0.088 mmol HCl). The resulting solution was kept at ambient temperature, placed in the refrigerator, then placed in the freezer with occasional scratching of the vial. After about 3 days in the freezer, crystallization had not occurred. Another 88 μL of a 1.0 M solution of hydrogen chloride in diethyl ether (0.088 mmol HCl) was added and attempts to induce crystallization as described above were repeated. After about 1 day in the freezer crystallization had not occurred. Three more 88 μL aliquots of a 1.0 M solution of hydrogen chloride in diethyl ether (0.088 mmol HCl each aliquot, total of 0.44 mmol HCl) were added, with subsequent attempts to crystallize, over the next 3 days. The final solution was kept in the freezer for about 3 days, during which time crystallization occurred. Filtration afforded 36 mg (23% yield) of R,R-reboxetine hydrochloride, Form B. Analytical data were obtained on the final product: the XRPD pattern was as shown in the top trace in FIG. 1, the DSC thermogram was consistent with that shown in FIG. 3, the TGA profile was consistent with that shown in FIG. 5, and the ¹H-NMR spectrum was as shown in FIGS. 7A-7G.

### Example 2

### Preparation of Form C of (R,R)-Reboxetine Hydrochloride

A solution of 3.38 g of R,R-reboxetine free base (shown by NMR to be contaminated with methanesulfonic acid) in 45 mL of ethyl acetate was prepared. Crystallization occurred from the solution. The resulting mixture was allowed to stand at ambient temperature for about one day, placed in a freezer for about 3 days, and filtered to give 0.97 g of crystalline R,R-reboxetine mesylate. Attempts were made to induce crystallization of additional mesylate salt from the filtrate, including addition of hexanes, seeding with R,R-reboxetine mesylate, cooling, and concentration by solvent evaporation. No additional crystalline material was obtained. The filtrate was dissolved in 32 mL of ethyl acetate and treated slowly with 7.7 mL of a 1.0 M solution of hydrogen chloride in diethyl ether. The resulting solution was stirred at ambient temperature for about 1 day, seeded with 4 mg of R,R-reboxetine hydrochloride, Form B (Example 1), and left at ambient temperature. A small amount of crystal growth was observed after 4 hours. The mixture was kept at ambient temperature for about 1 day after seeding, at which time the liquid was decanted from the solid. The liquid decanted was seeded with 3 mg of R,R-reboxetine hydrochloride, Form B (Example 1), and the resulting mixture was placed in the refrigerator for about 2 days. Some crystal growth was evident. The mixture was placed in the freezer for about 3 days and filtered to give 489 mg of R,R-reboxetine hydrochloride, Form C, as a light yellow solid. Analytical data were obtained on the final product: the XRPD pattern was as shown in the bottom trace in FIG. 2, the DSC thermogram was consistent with that shown in FIG. 4, the TGA profile was consist n with that shown in FIG. 6, and the ¹H-NMR spectrum was as shown in FIGS. 8A-8G.

### Example 3

### Preparation of Form B of (R,R)-Reboxetine Hydrochloride

The filtrate from isolation of R,R-reboxetine hydrochloride. Form C (Example 2) was concentrated under a stream of nitrogen and dissolved in 35 mL of ethyl acetate. The solution was extracted with a solution of 330 mg of sodium hydroxide in 25 mL of water. The water layer was extracted with two 10-mL portions of ethyl acetate. The ethyl acetate layers were combined and concentrated under a stream of nitrogen to give about 697 mg of a light brown oil. The oil was dissolved in 2 mL of ethyl acetate and treated with 2.22 mL of a 1.0 M solution of hydrogen chloride in diethyl ether. The resulting solution was seeded with a small amount of R,R-reboxetine hydrochloride, Form C (Example 2), kept in the refrigerator for about 3 days, and kept in the freezer for about 2 days. The solvent was decanted from the solid and the solid was dried under a stream of nitrogen to give 0.5 g of R,R-reboxetine hydrochloride, Form B. Analytical data were obtained on the final product: the XRPD pattern was as shown in the bottom trace in FIG. 1, the DSC thermogram was as shown in FIG. 3, the TGA profile was as shown in FIG. 5, and the ¹H-NMR spectrum was consistent with that shown in FIGS. 7A-7G.

### Example 4

### Preparation of Form C of (R,R)-Reboxetine Hydrochloride

A suspension of 757 mg (1.85 mmol) of R,R-reboxetine mesylate in 30 mL of ethyl acetate was extracted with a solution of 111 mg of sodium hydroxide in 10 mL of water. All of the solid dissolved. The layers were separated and the aqueous layer was extracted with three 30 mL portions of ethyl acetate. The ethyl acetate layers were combined, dried over magnesium sulfate, and concentrated to give an oil, which was dried under vacuum to give 530 mg (91% yield) of R,R-reboxetine free base as a yellow oil. The free base was dissolved in 1.2 mL of ethyl acetate and treated with 1.69 mL of a 1.0 M solution of hydrogen chloride in diethyl ether (1.69 mmol of HCl). Phase separation occurred to give a yellow oil. The oil was dissolved by addition of 0.8 mL of ethyl acetate. Solid precipitated from the solution. The slurry was stirred at ambient temperature in an open vial until enough solvent evaporated to reduce the volume by about ⅓. Filtration and washing of the solid with 5 mL of diethyl ether afforded 478 me, (81% yield) of solid R,R-reboxetine hydrochloride, Form C. Analytical data were obtained on the final product: the XRPD pattern was as shown in the top trace in FIG. 2, the DSC thermogram was as shown in FIG. 4, the TGA profile was as shown in FIG. 6, and the ¹H-NMR spectrum was consistent with that shown in FIGS. 8A-8G.

What is claimed is:

1. Form B (2R)-2-[(R)-(2-ethoxyphenoxy)-phenylmethyl]morpholine hydrochloride.

2. Form B (2R)-2-[(R)-(2-ethoxyphenoxy)-phenylmethyl]morpholine hydrochloride having substantially the same XRPD pattern as shown in FIG. 1.

3. A substantially enantiopure Form B (2R)-2-[(R)-(2-ethoxyphenoxy)-phenylmethyl]morpholine hydrochloride.

4. A substantially enantiopure Form B (2R)-2-[(R)-(2-ethoxyphenoxy)-phenylmethyl]morpholine hydrochloride having substantially the same XRPD pattern as shown in FIG. 1.

5. A pharmaceutical composition comprising Form B (2R)-2-[(R)-(2-ethoxyphenoxy)-phenylmethyl]morpholine hydrochloride according to claim 1.

6. A pharmaceutical composition comprising Form B (2R)-2-[(R)-(2-ethoxyphenoxy)-phenylmethyl]morpholine hydrochloride according to claim 2.

7. A pharmaceutical composition comprising the substantially enantiopure Form B (2R)-2-[(R)-(2-ethoxyphenoxy)-phenylmethyl]morpholine hydrochloride according to claim 3.

8. A pharmaceutical composition comprising the substantially enantiopure Form B (2R)-2-[(R)-(2-ethoxyphenoxy)-phenylmethyl]morpholine hydrochloride according to claim 4.

9. A method of treating and/or preventing a nervous system or pain disorder comprising administering a pharmaceutical composition comprising Form B (2R)-2-[(R)-(2-ethoxyphenoxy)-phenylmethyl]morpholine hydrochloride according to claim 5.

10. A method of treating and/or preventing a least one of depression, a nervous system disorder, or pain disorder comprising administering a pharmaceutical composition comprising Form B (2R)-2-[(R)-(2-ethoxyphenoxy)-phenylmethyl]morpholine hydrochloride according to claim 6.

11. A method of treating and/or preventing at least one of depression, a nervous system disorder, or pain disorder comprising administering a pharmaceutical composition comprising Form B (2R)-2-[(R)-(2-ethoxyphenoxy)-phenylmethyl]morpholine hydrochloride according to claim 7.

12. A method of treating and/or preventing at least one of depression, a nervous system disorder, or pain disorder comprising administering a pharmaceutical composition comprising Form B (2R)-2-[(R)-(2-ethoxyphenoxy)-phenylmethyl]morpholine hydrochloride according to claim 8.
